(19) United States
(12) Patent Application Publication
(10) Pub. No.: US 2021/0024596 A1
(43) Pub. Date: Jan. 28, 2021

(54) PRO-COAGULANT HISTONES

(71) Applicant: The University of Liverpool, Liverpool (GB)

(72) Inventors: Cheng Hock Toh, Liverpool (GB); Guozheng Wang, Liverpool (GB); Simon Abrams, Liverpool (GB)

(21) Appl. No.: 17/040,471

(22) PCT Filed: Mar. 26, 2019
(86) PCT No.: PCT/GB2019/050850

§ 371 Date: Sep. 22, 2020

(30) Foreign Application Priority Data
Mar. 26, 2018 (GB) 1804833.0

(51) Int. Cl. C07K 14/47; A61P 7/04

(52) U.S. Cl. CPC C07K 14/47; A61K 38/00; A61P 7/04

(57) ABSTRACT

The present invention relates to modified histone proteins for use in promoting coagulation, wherein the modified histone protein has reduced cytotoxicity as compared to a corresponding wild-type histone protein. The invention also relates to said modified histone proteins for use in promoting coagulation in the inhibition of bleeding and in promoting coagulation in a coagulation assay. In addition, the invention relates to a method of promoting coagulation in a subject in need thereof, the method comprising the step of providing the subject with said modified histone proteins. The invention also relates to an expression vector comprising a nucleic acid encoding said modified histone protein.

### FIELD OF THE INVENTION

The present invention relates to modified histone proteins for use in promoting coagulation. The invention also relates to modified histone proteins for use in promoting coagulation in inhibition of bleeding. Further, the invention also relates to modified histone proteins for use in promoting coagulation in a coagulation assay. The invention also relates to a method of promoting coagulation in a subject in need thereof, the method comprising the step of providing the subject with a modified histone protein. Moreover, the invention relates to a modified histone protein wherein the modified histone protein has reduced cytotoxicity as compared to a corresponding wild-type histone protein, as well as an expression vector comprising a nucleic acid encoding said modified histone protein.

### INTRODUCTION

Uncontrollable and excessive bleeding is often a major problem in surgery and trauma, as well as in a number of bleeding disorders, for example haemophilia A and B, and von Willebrand's disease. Haemophilia A and B are genetic diseases characterised in factor VIII deficiency and factor IX deficiency, respectively. Von Willebrand's disease is also a genetic disorder, caused by the lack of or an abnormal von Willebrand factor protein.

The coagulation cascade is a chain of interlinked reactions which stop bleeding. The cascade is made up of two pathways—the intrinsic and the extrinsic coagulation pathways. An intermediate event in the intrinsic pathway is the activation of factor IX to factor IXa, a reaction catalysed by factor XIa and calcium ions. Factor IXa then participates in the activation of factor X to factor Xa in the presence of factor VIIIa, phospholipids and calcium ions.

The extrinsic pathway involves plasma factors and other components present in tissue extracts. Factor VII participates in the extrinsic pathway of blood coagulation by converting (upon its activation to Vila) factor X to Xa in the presence of tissue factor and calcium ions. Factor Xa (along with factor Va as a co-factor) then converts prothrombin to thrombin in the presence of calcium ions and phospholipids. Thrombin, in turn, catalyses the conversion of fibrinogen to fibrin. Fibrin is an insoluble polymeric protein, which impedes the blood flow, and together with platelets forms what is known as “the haemostatic plug”, which is dissolved later by fibrinolytic processes.

Because the activation of prothrombin to thrombin is an event shared by both the intrinsic and extrinsic pathways, targeting this reaction so as to increase the formation of thrombin, and thereby increase the formation of fibrin, may be beneficial to prevent bleeding regardless of which pathway is defective.

Current treatments for bleeding disorders involve replacing missing coagulation factors or when this is not sufficient or involving multiple factors or when antibodies against coagulation factors arise, by-passing agents, such as activated prothrombin complex (which contains exogenously activated factor II, factor VII, factor IX and factor X), as well as recombinant activated factor VII. However, neither of these treatments are predictably effective, they have a high failure rate and may lead to fatal haemorrhage in some patients.

Consequently, there is a need in the art for improved agents and methods for promoting coagulation, and inhibiting bleeding.

### SUMMARY OF THE INVENTION

In a first aspect, the present invention relates to a modified histone protein for use in promoting coagulation, wherein the modified histone protein has reduced cytotoxicity as compared to a corresponding wild-type histone protein.

In one embodiment, there is provided a use of a histone protein or modified histone protein in promoting coagulation.

In a suitable embodiment, the modified histone is for use in promoting coagulation in inhibition of bleeding.

Accordingly, in a second aspect, the present invention relates to a modified histone protein for use in promoting coagulation in inhibition of bleeding, wherein the modified histone protein has reduced cytotoxicity as compared to a corresponding wild-type histone protein.

It will be appreciated that bleeding may be as a result of a coagulation disorder.

Thus, in a suitable embodiment, a modified histone protein for use according to the second aspect, may be for use in the treatment or prevention of a coagulation disorder.

In a third aspect, the present invention relates to a modified histone protein for use in promoting coagulation in a coagulation assay.

In a fourth aspect, the present invention relates to a method of promoting coagulation in a subject in need thereof, the method comprising the step of providing the subject with a therapeutically effective amount of a modified histone protein, wherein the modified histone protein has reduced cytotoxicity as compared to a corresponding wild-type histone protein.

In a suitable embodiment, the method is of promoting coagulation in inhibition of bleeding.

In a fifth aspect, the present invention relates to a modified histone protein wherein the modified histone protein has reduced cytotoxicity as compared to a corresponding wild-type histone protein.

In a sixth aspect, the present invention relates to an expression vector comprising a nucleic acid encoding a modified histone protein according to the fifth aspect.

In a seventh aspect, the present invention relates to a modified histone protein wherein the modified histone protein is at least 70% identical to one of the following sequences: SEQ ID NO. 5, SEQ ID NO. 4, SEQ ID NO. 3, SEQ ID NO 2, and SEQ ID NO. 1.

In an eighth aspect, the present invention relates to a modified histone protein wherein the modified histone protein is at least 70% identical to one of the following sequences: SEQ ID NO. 6, or SEQ ID NO. 7.

### BRIEF DESCRIPTION OF THE DRAWINGS

Embodiments of the invention are further described hereinafter with reference to the accompanying drawings, in which:

FIG. 1 Identification of prothrombin from plasma as a histone binding protein. (A) Shows Coomassie brilliant blue staining of histone-binding proteins (black circles) captured from plasma by histone-conjugated sepharose and separated by 2D gel. (B) A graph showing typical LC-MS peaks of a peptide (SEQ ID NO: 8) from trypsin-digested spot from 2D gel. (C) Shows results of western blotting of isolated histone-binding proteins using anti-prothrombin antibody with commercial prothrombin (ProT) as a positive control. Arrow indicates the full length of prothrombin. (D) Shows results of a gel overlay assay: equal molar concentrations (2 μmol/L) of recombinant individual human histones were subjected to SDS-PAGE and probed with HRP-conjugated prothrombin (upper panel). Lower panel: Coomassie blue stained gel to demonstrate equal loading of proteins.

FIG. 2 Identification of prothrombin from plasma as a histone binding protein. A, B and C show surface plasmon resonance (SPR) curves for calculating Kds of prothrombin with histone H2B, H3 and H4 respectively. Each experiment was repeated 3 times and a typical experiment is presented.

FIG. 3 Alignment of peptides identified by mass spectrometry. The alignments of identified peptides with the sequences of prothrombin (ProT) fragment 1 (upper) (SEQ ID NO: 9) and 2 (lower) (SEQ ID NO: 10) are presented to confirm that the protein isolated is prothrombin.

FIG. 4 Effect of individual histones on prothrombin cleavage. (A) Coomassie blue stained gel shows the cleavage of prothrombin (ProT) by FXa in the presence of individual histones after 60 min incubation with calcium (5 mmol/L) at 37° C. (B) Densitometric quantification of the percentage of prothrombin digested over time. Mean curves from 3 independent experiments are shown. (C) Effect of histones on prothrombin digestion without FXa after 60 min incubation with calcium (5 mmol/L) at 37° C. Densitometric quantification of prothrombin bands represented as Mean±SD from 3 independent experiments. *Student t-test compares prothrombin cleavage by histones with and without FXa (P<0.05).

FIG. 5 Effect of individual histones on thrombin generation. (A) Thrombin generation from FXa activation of prothrombin in the presence of individual histones and calcium (5 mmol/L) with typical curves from 3 independent experiments presented. (B) Mean±SD of peak thrombin generation (nmol/L) from 3 independent experiments. *Student t-test P<0.05 compared to that in the absence of histones.

FIG. 6 Effect of anti-histone reagents on histone-induced prothrombin cleavage. Prothrombin cleavage by FXa in the presence of H3 or H4 without or with ahscFv (A) or heparin (6 μmol/L) (B) following 60 min incubation with calcium (5 mmol/L) at 37° C. Percentages were calculated from Coomassie blue stained gels from 3 independent experiments (Mean±SD). *Student t-test shows significant increase compared with prothrombin+FXa alone untreated by histones (UT) (P<0.05). #significant reduction (P<0.05) when comparing prothrombin cleavage in the absence or presence of anti-histone-reagents.

FIG. 7 Effect of anti-histone reagents on histone-induced thrombin generation. The effects of ahscFv (A) and heparin (B) on thrombin generation presented as Mean±SD of peak thrombin (nmol/L). *Student t-test shows significant increase compared with prothrombin+FXa alone untreated by histones (UT) (P<0.05). #significant reduction (P<0.05) when comparing peak thrombin in the absence or presence of anti-histone-reagents.

FIG. 8 Prothrombin cleavage in the presence and absence of calcium. Coomassie blue stained gel shows the cleavage of prothrombin (ProT) by FXa in the presence of H4 after 60 min incubation with different buffers at 37° C. Prothrombin cleavage was performed in 20 mmol/L HEPES, 150 mmol/L NaCl (Control), in the presence of calcium (5 mmol/L final concentration) or EGTA (2 mmol/L final concentration). The band below H4 is likely due to thrombin-induced H4 degradation as previously reported.

FIG. 9 Histone H3 and H4 bind to fragment 1 and fragment 2 of prothrombin. Computer prediction using the ZDOCK server shows that H3 (A), H4 (B) and H2B (C) bind to prothrombin. Specifically, H3 and H4 recognize prothrombin fragments 1 and 2, whilst H2B recognizes the protease domain. (D) Schematic representation of fragment 1, 2 and protease domains of prothrombin.

FIG. 10 Histone H3 and H4 binding to fragment 1 and fragment 2 of prothrombin competes with FVa. (A) Coomassie blue stained SDS-PAGE gel showing purified prothrombin fragments 1 and 2 produced in BL21 bacteria. (B) Binding affinities (Kd) of H3 and H4 to prothrombin fragments 1 and 2 by Surface plasmon resonance (SPR) kinetic assay. (C) Total thrombin generation from FXa activation of prothrombin in the presence of FVa with or without phospholipids (PL). All reactions were performed for 90 seconds in the presence of calcium (5 mmol/L) and terminated by the addition of EDTA (10 mmol/L). Mean±SD from 3 independent experiments. *Student t-test shows significant decrease in prothrombin cleavage in the absence of phospholipids (P<0.05). (D) Thrombin generation during 90 seconds activation of prothrombin by FXa with FVa, H3 or H4 in the presence of calcium (5 mmol/L) and absence of phospholipids. Mean±SD from 3 independent experiments. *Student t-test shows significant increase in total thrombin generation compared to FVa (P<0.05). (E) Thrombin generation in re-calcified FV-deficient PPP±H4 (50 μg/mL).

FIG. 11 Prothrombin cleavage assays. Time courses of prothrombin cleavage in the presence of FXa and 50 μg/mL histone H3 (A) or H4 (B) in vitro. (C) and (D) correspond to (A) and (B) respectively, but without FXa.

FIG. 12 Prothrombin cleavage assays. (A) Same cleavage assay as FIG. 11 but without histones and FXa. (B) Same cleavage assay as FIG. 11 but without histones. All reactions were performed in the presence of calcium (5 mmol/L) at 37° C.

FIG. 13 Histones enhance thrombin generation in the absence of FV and phospholipids. Prothrombin cleavage by FXa in the presence of FVa, H3 or H4 with (A) or without (B) phospholipids (40% PC; 20% PS; 40% PE) after 60 min incubation with calcium (5 mmol/L) at 37° C. (C) Densitometric quantification of prothrombin bands from gels (A and B) and represented as Mean±SD from 3 independent experiments. *Student t-test shows significant decrease in prothrombin cleavage in the absence of phospholipids (P<0.05). (D) Total thrombin generation from FXa activation of prothrombin in the presence of FVa with or without phospholipids (PL). All reactions were performed for 90 seconds in the presence of calcium (5 mmol/L) and terminated by the addition of EDTA (10 mmol/L). Mean±SD from 3 independent experiments. *Student t-test shows significant decrease in prothrombin cleavage in the absence of phospholipids (P<0.05).

FIG. 14 Histones enhance thrombin generation in the absence of FV and phospholipids. Thrombin generation during 90 seconds activation of prothrombin by FXa with FVa, H3 or H4 in the presence of calcium (5 mmol/L) and absence of phospholipids. Mean±SD from 3 independent experiments. *Student t-test shows significant increase in total thrombin generation compared to FVa (P<0.05).

FIG. 15 Surface plasmon resonance kinetic assays. Typical curves for calculating binding affinities of H3 and H4 to prothrombin fragment 1 (A and B, respectively) and fragment 2 (C and D).

FIG. 16 Effects of histone H4 on thrombin generation in factor deficient plasmas. Thrombin generation of FV, FX and FII (ProT) deficient plasma after re-calcifying in the presence and absence of histone H4 was performed. Means±SD of peak thrombin from 3 independent experiments are presented. *p<0.01 when compared to that without histones.

FIG. 17 Histones reduce the demand for FXa to enhance thrombin generation and clot formation. (A) Thrombin generation from FXa activation of prothrombin in the presence different concentrations of FXa±H4 (50 μg/mL). (B) Typical thrombin generation curve in re-calcified FX-deficient PPP±FXa (500 μmol/L)±histones (50 μg/mL). (C) Clot formation of re-calcified (12.5 mmol/L) FX-deficient plasma±H4 (50 μg/ml) supplemented with either 500 pM, 1000 pM or 5000 pM FXa.

FIG. 18 Histones reduce the demand for FXa to enhance thrombin generation and clot formation in haemophilia plasma. (A) FXa concentrations following re-calcification of normal, FVIII and FIX-deficient PPP. (B) Histone H4 (50 μg/ml) dramatically boosts thrombin generation in FVIII and FIX-deficient plasma. (C) 50 μg/ml H4 (solid line lines) induced clot formation in FVIII and FIX-deficient plasma after re-calcification although slower than normal plasma (black), much improved comparing to histone absence (dotted lines), which did not clot.

FIG. 19 Effect of phospholipids on histone enhanced thrombin generation. Thrombin generation from FXa-mediated prothrombin activation in the presence of 50 μg/mL H3 (A) or H4 (B) with or without phospholipids (5 μmol/L). All reactions were performed for 90 seconds in the presence of calcium (5 mmol/L) and terminated by the addition of EDTA (10 mmol/L). Mean±SD from 3 independent experiments.

FIG. 20 Prothrombin F1 and F2 alleviate histone-induced systemic coagulation activation in vivo. C57BL/6 male mice were anesthetized and infused with different doses of histones through tail veins. One hour after infusion, blood were taken for platelet count, prothrombin time (PT) and concentrations of fibrinogen, thrombin-antithrombin complexes (TAT) and D-dimer (A). DIC scores were also calculated (A). (B) Lung sections were immunohistochemically stained with anti-fibrin antibody and typical images are presented. Arrows indicate thrombi. Bar=50 μm. (C) Platelet counts, TAT and fibrinogen levels following F1F2 infusion±histones.

FIG. 21 Histone-induced prothrombin activation can directly enhance thrombin generation when the intrinsic pathway amplification loop is compromised by Factor VIII deficiency.

FIG. 22 Histone-enhanced thrombin generation in platelet-poor plasma (PPP) and platelet-rich plasma (PRP). Thrombin generation in PPP or PRP+H4±prostaglandin E1 (PGE1) (10 μmol/L) was performed. Typical thrombin generation curves were presented.

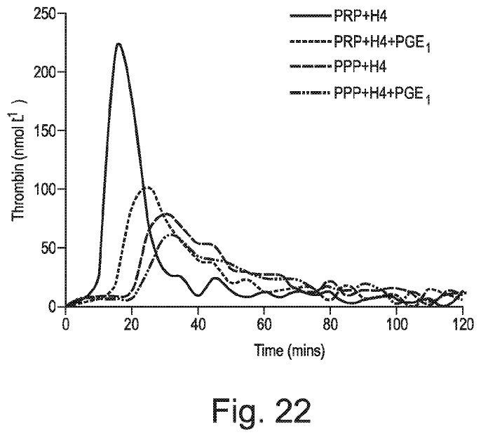

FIG. 23 Truncated modified histone proteins H3C and H4C enhance thrombin generation. (A) Schematic representation of both histone H3 (upper) and H4 (lower) proteins. Demonstrating where both the N-(start) and C-terminal (end) regions are located within the total protein structure. (B) Thrombin generation from FXa activation of prothrombin in the presence of H3C and H4C-terminal peptides and calcium (5 mmol/L). (C) Mean thrombin generation in normal platelet-poor plasma (PPP) incubated with either H4N or H4C-terminal peptides and calcium.

FIG. 24 Biological function of exemplary modified histone proteins. (A) Results of a cell viability assay which show that modified histone proteins, H3C and H4C, have a significantly reduced cytotoxicity as compared to the corresponding wild-type histones. (B) Results of a gel overlay assay, demonstrating H3C and H4C binding to prothrombin: commercial prothrombin (ProT) was subjected to SDS-PAGE and probed with either HRP-conjugated H3C (left panel) or HRP-conjugated H4C (middle panel) proteins. Left panel: Coomassie blue stained gel.

### DETAILED DESCRIPTION OF THE INVENTION

The present invention is based upon the inventors' surprising finding that histone proteins may be modified in a way that reduces their cytotoxicity yet maintains their ability to promote coagulation and ultimately also inhibit bleeding.

The inventors have found that histone proteins can replace factor Va in the prothrombinase complex, and together with factor Xa (FXa) form a complex with prothrombinase activity. The inventors have shown that such a complex is capable of converting prothrombin to thrombin, and ultimately promoting coagulation.

Moreover, the inventors have found that histone proteins, which are naturally cytotoxic may be modified such that their cytotoxicity is reduced without compromising their ability to convert prothrombin to thrombin. Such modified histone proteins may be used as therapeutic agents.

As touched upon above, thrombin is an important component of the intrinsic and extrinsic coagulation pathways. In addition to converting fibrinogen to fibrin, thrombin initiates a positive feedback loop which results in further activation of factors V, VIII, XI and IX to generate more thrombin. As a result, even more fibrinogen is converted to fibrin and clot formation is amplified.

Histone proteins (H1, H2A, H2B, H3, H4 and H5) are a family of proteins, which are normally found within the cell nucleus, and due to their positive charge, bind DNA to form chromatin. They are also important for extra-cellular host defence mechanisms. Specifically, upon pathogen induced cell damage, histones are released into the extracellular space where they are directly toxic to the invading pathogens. Additionally, histones form neutrophil extracellular traps (NETs) which serve to immobilise pathogens. Unfortunately, histones are also very toxic to host cells.

Some histones (such as H3 and H4) have been also previously shown to bind and auto-activate prothrombin, resulting in the formation of thrombin. However, as this was only observed in buffer conditions and not in plasma, thus it has previously been unclear if in vivo histones have this function. Additionally, the auto-activation of prothrombin by histones was also found to be slow (taking up to 8 hours). This meant that due to their cytotoxicity as well as slow pro-coagulant activity histone proteins were not considered to be useful in a clinical setting, especially in the context of excessive bleeding where fast action is required.

Therefore, although the role of histones in the conversion of prothrombin to thrombin has been previously described, the therapeutic use of histone proteins for promoting coagulation has not been previously thought to be possible.

Advantageously, the inventors have found that histone proteins can be modified such that they have a reduced or substantially no cytotoxic effect. Furthermore, the inventors have found that modified histone proteins maintain their ability to promote coagulation. Such modified histone proteins have a clear therapeutic utility, and give rise to the first, second, and fourth aspects of the invention.

It will be appreciated, that whilst reduced cytotoxicity may be important in the therapeutic applications of histone proteins, in in vitro methods this might not necessarily be the case. Such non-therapeutic uses of modified histone proteins give rise to the third aspect of the invention.

Furthermore, without wishing to be bound by any hypothesis, the inventors believe that the modified histone proteins of the invention, due to their small size, are less likely to be immunogenic as compared to some of the current treatments (for example recombinant activated factor VII). As such, the inventors believe that the proteins of the invention are a promising alternative treatment for patients who have developed antibodies to other coagulation disorder treatments.

The invention, and certain terms used in the disclosure of the present invention, will now be defined and described further.

### A Modified Histone Protein

The present invention is based upon the finding that modified histone proteins are able to promote coagulation.

The term “histone” or “histone protein” as referred to herein indicates a protein that is usually found in a eukaryotic cell nucleus and responsible for packaging and ordering DNA into a structural unit called a nucleosome.

The term “wild-type histone protein” as used herein refers to a naturally occurring form of a histone protein (i.e. a histone protein which has not been modified) or a naturally occurring variant thereof.

References to a “corresponding wild-type histone protein” as used herein indicate a comparison of a modified histone protein with the same histone protein in its naturally occurring form which has not been modified. For example, when considering a modified H3 histone protein, the relevant corresponding wild-type histone protein is histone H3 which may be as defined in SEQ ID NO. 4.

Known naturally occurring histone proteins are histone H1 also known as histone H5 in some species (SEQ ID NO. 1), histone H2A (SEQ ID NO. 2), histone H2B (SEQ ID NO. 3), histone H3 (SEQ ID NO. 4), histone H4 (SEQ ID NO. 5).

Naturally occurring variants of said histone proteins are also known. Examples of naturally occurring variant histones are follows: H3.1, H3.2, TS H3.4, H3.3, centromeric H3, H2AZ, H2AB, H2AW, H2AL, H2AP, H2A1, H2AX, H2B1, H2BW, and H2BE. Further naturally occurring histone proteins and variant histone proteins may be found in the database “HistoneDB 2.0”.

Suitably, a naturally occurring variant of a histone protein may be at least 70%, at least 75%, at least 80%, be at least 85%, at least 90%, at least 95%, at least 96%, at least 97%, at least 98%, at least 99%, or more, identical to a sequence of naturally occurring histone proteins, for example histone H3 (SEQ ID NO: 4) or histone H4 (SEQ ID NO: 5).

“Modified” as used herein refers to a histone protein that has been changed such that its amino acid sequence is not the same as the amino acid sequence of the corresponding wild-type histone protein. It will be appreciated that a modified histone protein is, by definition, not a wild-type histone protein.

The sequence of the modified histone protein may be changed by man-made modifications or otherwise. Such modifications may include deletions (for example single amino acid deletions, deletions of 2 or more contiguous amino acids, and/or truncations); inversions, substitutions, repeats, reversals; amino acid modifications including tagging, phosphorylation, methylation and biotinylation, and the like.

One or more modifications may be present in a modified histone protein sequence.

One or more different types of modification may be present in a modified histone protein sequence.

Suitably, the modified histone protein may be a fragment and/or may comprise one or more amino acid sequence mutations.

Suitably, the modified histone proteins described herein have reduced cytotoxicity in comparison with the corresponding wild-type histone protein. “Reduced cytotoxicity” is defined hereinbelow.

In a suitable embodiment, a modified histone protein has been modified to reduce its cytotoxicity. Suitably, the modified histone protein has been modified to remove one or more cytotoxic portions of its amino acid sequence.

Suitably, the modified histone protein has been modified to reduce its cytotoxicity yet retain its ability to promote coagulation. Suitably, the modified histone protein has been modified to reduce its cytotoxicity yet retains at least 50%, at least 60%, at least 70%, at least 80%, at least 90%, at least 95%, at least 97%, at least 99% at least 100% of its ability to promote coagulation in comparison with a corresponding wild-type histone protein.

The term “fragment” as used herein refers to an incomplete histone protein as compared to the corresponding wild-type histone protein. Such fragments may include a truncated form of a wild-type histone protein, or a wild-type histone protein that has one or more domains, sections, or parts of its amino acid sequence missing, or that have been removed.

Suitably, the modified histone protein is a fragment of the corresponding wild-type histone protein. Suitably the modified histone protein is a truncated histone protein. Suitably, the modified histone protein consists of a truncation of the corresponding wild-type histone protein amino acid sequence.

In a suitable embodiment a fragment may share at least 70%, at least 75%, at least 80%, at least 85%, at least 90%, at least 95%, at least 99%, or more, sequence identity with the portion of the wild-type sequence which it corresponds to. More suitably, a fragment may share a 100% sequence identity with the portion of the wild-type sequence which it corresponds to.

Suitably such a fragment may, consist of approximately 20% to 95%, or 30% to 90%, or 40% to 60%, or 45% to 55% of the total length of the corresponding wild-type histone amino acid sequence. Suitably such a fragment may consist of approximately 30%-50% of the total length of the corresponding wild-type histone amino acid sequence.

Suitably, the modified histone protein has been modified to remove at least a part of the N-terminal and/or C-terminal region.

In one embodiment, the modified histone protein is a truncated histone protein. Suitably, the truncated histone proteins lacks all or part of the N-terminal tail region.

Suitably, the modified histone protein may be a truncated histone protein lacking a part of the N-terminal tail region and a part of the C-terminal region and further comprising multiple amino acid sequence mutations.

In one embodiment, the modified histone protein is a truncated histone protein lacking a part of the C-terminal region.

In an embodiment where the modified histone protein is a truncated histone protein lacking a part of the C-terminal region, the modified histone protein may consist of amino acids 1-120, 1-110, 1-100, 1-90, 1-80, 1-70, 1-60, 1-50, or 1-40 of the corresponding wild-type histone amino acid sequence, and any integer value therebetween.

In an embodiment where the modified histone protein is a truncated histone protein lacking a part of the N-terminal tail region, the modified histone protein may consist of amino acids 40 onwards, 50 onwards, 60 onwards, 70 onwards, 80 onwards, 90 onwards, 100 onwards, 110 onwards, or 120 onwards of the corresponding wild-type histone amino acid sequence, and any integer value therebetween.

Suitably, the modified histone protein may be a truncated histone protein lacking part or all of the C-terminal region of SEQ ID NO. 5, SEQ ID NO. 4 SEQ ID NO. 3, SEQ ID NO. 2, or SEQ ID NO. 1.

Suitably, the modified histone protein may be a truncated histone protein lacking amino acids 40 onwards, 50 onwards, 60 onwards, 70 onwards, 80 onwards, 90 onwards, 100 onwards, 110 onwards, 120 onwards or any integer value therebetween of SEQ ID NO. 5, SEQ ID NO. 4, SEQ ID NO. 3, SEQ ID NO. 2, or SEQ ID NO. 1.

Suitably, the modified histone protein may be a truncated histone protein lacking amino acids 1-120, 1-110, 1-100, 1-90, 1-80, 1-70, 1-60, 1-50, 1-40 or any integer value therebetween of SEQ ID NO. 5, SEQ ID NO. 4, SEQ ID NO. 1, SEQ ID NO. 2, or SEQ ID NO. 3.

Suitably, the modified histone protein may comprise one or more amino acid sequence mutations (such mutations being assessed with reference to the corresponding wild type histone protein amino acid sequence).

Suitably the mutations are selected from one of: deletions, inversions, substitutions, repeats, or reversals of the histone protein amino acid sequence. In one embodiment, the modified histone protein comprises one or more point mutations. In one embodiment, the modified histone protein comprises one or more substitution mutations.

Suitably, the modified histone protein comprises one or more amino acid mutations to remove positively charged amino acid residues. Suitably, the positively charged amino acid residues are removed by substitution mutations. Suitably, the positively charged amino acid residues are removed by substitution with a neutral or negatively charged amino acid residue. Positively charged amino acid residues which may be substituted include: lysine, arginine, and/or histidine.

Suitably the modified histone protein comprises one or more lysine substitution mutations.

Suitably, the modified histone protein comprises one or more lysine to alanine substitution mutations.

Merely by way of example, in the context of histone H4 (as set out in SEQ ID NO: 5), the modification may a substitution of one or more amino acids selected from the group consisting of Lys31, Arg35, Arg39 and Arg45.

Suitably, the modified histone protein may comprise at least 1, at least 2, at least 3, at least 4, at least 5, at least 6, at least 7, at least 8, at least 9, at least 10, at least 20, at least 30, at least 40, or more amino acid mutations, which may suitably be substitutions, when compared to the corresponding wild-type histone protein amino acid sequence.

Suitably, the modified histone protein may comprise up to 50, up to 40, up to 30, up to 20, up to 10, up to 9, up to 8, up to 7, up to 6, up to 5, up to 4, up to 3, up to 2, or 1 amino acid mutations, which may suitably be substitutions when compared to the corresponding wild-type histone protein amino acid sequence.

Suitably the modified histone protein comprises up to 30 amino acid substitutions when compared to the corresponding wild type histone protein amino acid sequence.

In one embodiment, the modified histone protein comprises between 5 to 20 amino acid substitutions when compared to the corresponding wild type histone protein amino acid sequence.

In one embodiment, the modified histone protein comprises 4 amino acid substitutions when compared to the corresponding wild type histone protein amino acid sequence.

In one embodiment, the modified histone protein comprises 3 amino acid substitutions when compared to the corresponding wild type histone protein amino acid sequence.

In one embodiment, the modified histone protein comprises 2 amino acid substitutions when compared to the corresponding wild type histone protein amino acid sequence.

In one embodiment, the modified histone protein comprises 1 amino acid substitution when compared to the corresponding wild type histone protein amino acid sequence.

Suitably, the positively charged amino acid residues which are substituted are located in an exposed part of the histone protein structure. Suitably, the positively charged amino acid residues which are substituted are outside of any tertiary structure in the histone protein such as alpha helices or beta sheets. Suitably, the positively charged amino acid residues which are substituted are outside of the histone protein core domain. Suitably, the positively charged amino acid residues which are substituted are not located in the alpha helices of the histone protein structure. Suitably, the positively charged amino acid residues which are substituted are located in the N-terminal tail region of the histone protein.

Suitably, fragments of histone proteins and histone proteins comprising amino acid sequence mutations share the biological activity of the corresponding wild-type histone proteins. In particular, the fragmented or mutated versions should share at least the ability to promote coagulation.

Suitably, the modified histone protein retains at least two alpha helices, suitably three alpha helices. Suitably, the modified histone protein retains a globular/core domain.

In accordance with the invention, the modified histone protein is at least 70% identical to one of the following sequences: SEQ ID NO. 5, SEQ ID NO. 4, SEQ ID NO. 3, SEQ ID NO 2, and SEQ ID NO. 1.

Suitably, the modified histone protein comprises an amino acid sequence which is at least 75%, at least 80%, at least 85%, at least 90%, at least 95%, at least 99% identical to one of the following sequences: SEQ ID NO. 5, SEQ ID NO. 4, SEQ ID NO. 3, SEQ ID NO 2, and SEQ ID NO. 1.

Suitably, the modified histone protein is not a wild-type histone protein.

In accordance with the invention, the modified histone protein comprises an amino acid sequence which is at least 70% identical to a sequence selected from the group consisting of SEQ ID NO. 6, and SEQ ID NO. 7.

Suitably, the modified histone protein may be at least 75%, or at least 80%, at least 85%, at least 90%, at least 95%, at least 99% identical to a sequence selected from the group consisting of SEQ ID NO. 6, and SEQ ID NO. 7.

In one embodiment, the modified histone protein consists of an amino acid sequence which is at least 70%, at least 75%, or at least 80% identical to a sequence selected from the group consisting of SEQ ID NO. 6, and SEQ ID NO. 7. Suitably the modified histone protein consists of an amino acid sequence which is at least 85%, at least 90%, at least 95%, at least 99% identical to a sequence selected from the group consisting of SEQ ID NO. 6, and SEQ ID NO. 7.

In one embodiment, the modified histone protein consists of one of the following sequences: SEQ ID NO. 6, or SEQ ID NO. 7.

### Reduced Cytotoxicity

In some embodiment, the present invention relates to modified histone proteins having reduced cytotoxicity as compared to a corresponding wild-type histone protein.

The term “cytotoxicity” refers to the toxicity of a histone protein, modified or not, to living cells, either in vivo or in vitro. Merely by way of example, toxicity to living cells may be measured by contacting living cells with the histone protein for a period of time and determining the number of living cells before and after the contact has been made, and calculating the difference in this number. If the number of cells that are alive has fallen in this time by a statistically significant amount, then the agent is generally regarded as cytotoxic. If the number of cells that are alive does not fall by a statistically significant amount, then the agent is generally regarded as non-cytotoxic. Other methods of determining cytotoxicity will be known to those skilled in the art.

Therefore “reduced cytotoxicity” as used herein means that the modified histone protein is less cytotoxic to living cells than a corresponding wild-type histone protein. Suitably, this is determined by directly comparing the cytotoxicity of the relevant modified histone protein to a corresponding wild-type histone protein in a toxicity assay.

For example, a typical toxicity assay may be conducted as demonstrated in the examples herein by culturing endothelial cells with 20 μg/ml of the relevant histone protein, modified or not, for 1 hour at 37° C. and under 5% CO₂. Then determining cell viability by staining the cells with propidium iodide and quantifying the number of alive cells using FACS. Cell viability is then expressed as a percentage of the untreated cells which is set to 100%.

Suitably the modified histone proteins have a cytotoxicity of less than 60%, less than 50%, less than 40%, less than 30%, less than 20%, suitably less than 15%, suitably less than 10%, suitably less than 8%, suitably less than 5%, suitably less than 3%, suitably less than 1%, or less as compared to a corresponding wild-type histone. Indeed, the modified histone proteins of the invention may be non-cytotoxic.

In other words, it can be said that the modified histone proteins maintain a cell viability to a greater extent than wild-type histone proteins. Suitably, modified histone proteins maintain the viability of over 80%, suitably over 85%, suitably over 90%, suitably over 92%, suitably over 95%, suitably over 97%, suitably over 99%, suitably 100% of cells brought into contact with the modified histone protein. More suitably, the modified histone proteins maintain a cell viability of between 99% and 100% of cells brought into contact with the modified histone protein.

Suitably, cytotoxicity and/or cell viability are calculated after culturing the modified histone protein for a period of time with live cells.

Suitably, cytotoxicity and/or cell viability may be calculated after culturing 20 μg/ml of the modified histone protein with live cells for 1 hour at 37° C. with less than 5% CO₂.

### Promoting Coagulation

By “promotion of coagulation” we mean increasing coagulation as compared to a suitable control.

In a suitable embodiment, coagulation may be increased by upregulating the clotting cascade. Suitably, the clotting cascade may be upregulated by increasing the amount or activity of one or more components of the clotting cascade. It will be appreciated that increasing the activity of one or more components of the clotting cascade may result in a decrease in the reaction time of one or more reactions of the clotting cascade. Suitably, the component and/or the reaction of the clotting cascade may be one of the intrinsic and/or extrinsic pathway.

Suitably, coagulation may be promoted ex vivo (for example in vitro) or in vivo.

An increase in viscosity of a blood or plasma sample may be taken as an indication of coagulation occurring in the sample. Suitable methods for determining viscosity will be known to those skilled in the art.

In a suitable embodiment, coagulation may be considered to be promoted if viscosity of blood and/or plasma is increased by at least 10%, at least 20%, at least 30%, at least 40%, at least 50%, at least 60%, at least 70%, at least 80%, or more as compared to a reference value. In a suitable embodiment, coagulation may be considered to be promoted if the rate of active thrombin generation is increased by at least 10%, at least 20%, at least 30%, at least 40%, at least 50%, at least 60%, at least 70%, at least 80%, or more as compared to a reference value.

In a suitable embodiment, coagulation may be considered to be promoted if the amount of thrombin is increased by at least 10%, at least 20%, at least 30%, at least 40%, at least 50%, at least 60%, at least 70%, at least 80%, or more as compared to a reference value.

In a suitable embodiment, coagulation may be considered to be promoted if prothrombin cleavage is increased by at least 10%, at least 20%, at least 30%, at least 40%, at least 50%, at least 60%, at least 70%, at least 80%, or more as compared to a reference value.

In a suitable embodiment, coagulation may be considered to be promoted if the rate of fibrin generation and/or the amount of fibrin is increased by at least 10%, at least 20%, at least 30%, at least 40%, at least 50%, at least 60%, at least 70%, at least 80%, or more as compared to a reference value.

Suitably, coagulation may be considered to be promoted if the amount and/or activity of a component of the intrinsic and/or extrinsic coagulation pathways selected from the group consisting of thrombin (activated tissue factor II), prothrombin (tissue factor II), fibrinogen (tissue factor I), fibrin (activated tissue factor I), tissue factor III, tissue factor V, activated tissue factor V, tissue factor VII, activated tissue factor VII, tissue factor VIII, activated tissue factor VIII, tissue factor IX, activated tissue factor IX, tissue factor X, activated tissue factor X, tissue factor XI, activated tissue factor XI, tissue factor XII, activated tissue factor XII, tissue factor XIII, and activated tissue factor XIII is increased. An amount and/or activity is considered to be increased if it is at least 10%, at least 20%, at least 30%, at least 40%, at least 50%, at least 60%, at least 70%, at least 80%, or more as compared to a reference value.

A suitable reference value may be obtained from a sample or subject in the absence of a histone modified protein. The sample may be a blood and/or plasma sample. A skilled person will be able to determine a suitable reference value without difficulty.

Other methods by which it can be determined whether coagulation has been promoted will be known to those skilled in the art.

### Inhibition of Bleeding

It will be appreciated that in vivo promotion of coagulation may ultimately inhibit bleeding. Thus, some of the uses and methods of the invention relate to promoting coagulation in inhibition of bleeding.

Suitably, bleeding may be spontaneous or non-spontaneous.

Spontaneous bleeding refers to bleeding that does not occur as a result of trauma. Suitable, spontaneous bleeding may be due to a coagulation disorder. Spontaneous bleeding may occur in any part of the body, for example nose, mouth, or digestive tract.

Non-spontaneous bleeding is caused by trauma. The term “trauma” as used herein refers to a physical injury of one or more body parts. By way of example, trauma may be caused by an accident, physical violence, surgery and/or childbirth. It will be appreciated that whilst non-spontaneous bleeding may occur in any subject, subjects with a coagulation disorder are at a greater risk of bleeding as a result of trauma.

Thus in a suitable embodiment, the modified histone proteins may be for use in the treatment or prevention of a coagulation disorder.

Alternatively, or additionally, the modified histone proteins may be for use in the treatment or prevention of trauma induced bleeding.

Inhibition of bleeding may be assessed with reference to the amount of blood lost due to spontaneous and/or non-spontaneous bleeding, or with reference to the time required to stop spontaneous and/or non-spontaneous bleeding. The amount of lost blood, or time it takes to stop bleeding may be assessed with reference to a suitable control, for example an individual without a coagulation disorder.

In a suitable embodiment, bleeding may be considered to be inhibited if it the amount of blood lost is reduced by at least 10%, at least 20%, at least 30%, at least 40%, at least 50%, at least 60%, at least 70%, at least 80%, at least 90%, or more as compared to a control.

In a suitable embodiment, bleeding may be considered to be inhibited if the time required to stop spontaneous and/or non-spontaneous bleeding is reduced by at least 10%, at least 20%, at least 30%, at least 40%, at least 50%, at least 60%, at least 70%, at least 80%, at least 90%, or more as compared to a control.

In a suitable embodiment, bleeding may be considered to be inhibited if it the rate of bleeding is reduced by at least 10%, at least 20%, at least 30%, at least 40%, at least 50%, at least 60%, at least 70%, at least 80%, at least 90%, or more as compared to a control.

### A Coagulation Disorder

The term “coagulation disorder” as used herein, refers to a condition in which there is a haemostatic imbalance. Such a haemostatic imbalance may result in excessive bleeding in a subject, and/or increased predisposition to excessive bleeding.

Suitably, a coagulation disorder may be caused by a clotting factor deficiency, a clotting protein deficiency, a defective platelet function and/or platelet deficiency, and/or overdevelopment of circulating anticoagulants and/or excessive fibrinolysis. In a suitable embodiment, a coagulation disorder may be genetic or acquired. Suitably, genetic coagulation disorders may be inherited.

A coagulation disorder caused by a clotting factor deficiency may be selected from the group consisting of haemophilia A, haemophilia B, factor I deficiency, factor H deficiency, factor V deficiency, factor VII deficiency, factor X deficiency, factor XI deficiency, factor XII deficiency and factor XIII deficiency.

A coagulation disorder caused by a clotting protein deficiency may be, for example, von Willebrand's disease.

A coagulation disorder caused by a defective platelet function and/or platelet deficiency may be, for example, selected from the group consisting of congenital conditions—Bernard Soulier syndrome, Glanzmann's thrombasthenia and platelet storage pool disorder, or acquired conditions such as immune thrombocytopaenic purpura (ITP).

An acquired coagulation disorder is any coagulation disorder which is not directly caused by an underlying genetic defect. Suitably, acquired enhanced complement activity is caused by disease. An acquired coagulation disorder may be as a result of a different (primary) disorder, or may be induced. A coagulation disorder caused by a different (primary) disorder, may be referred to as a secondary coagulation disorder.

By way of example, a primary disorder, which may lead to a secondary coagulation disorder, may be selected from the group consisting of liver disease (acquired or genetic), renal disease (acquired or genetic), multiple abnormalities in blood vessels that include hereditary haemorrhagic telangiectasia (HHT), hypergammaglobulinemia (for example multiple myeloma, or Waldenstrom macroglobuliaemia), systemic amyloidosis and vitamin K deficiency, trauma, or haemorrhage, in particular post-partum haemorrhage. Other examples of primary disorders which may result in an acquired coagulation disorder will be known to those skilled in the art.

By way of example, a primary coagulation disorder can include hyperfibrinoloysis which may arise secondary to trauma and post-partum haemorrhage.

By way of example, an induced coagulation disorder, may be due to exposure to drugs, alcohol and/or malnutrition. Drugs which may induce a coagulation disorder include anticoagulant drugs (such as warfarin, dabigatran, rivaroxaban, apixaban, edoxaban), anti-thrombotic drugs, (for example aspirin), antibiotics, clopidogrel, IIbIIIa inhibitors, clotting factors, and/or fibrinolytic agents that include tissue-type plasminogen activator (tPA), urokinase and streptokinase. Other examples of induced coagulation disorders will be known to those skilled in the art.

By way of example, an induced coagulation disorder can include acquired haemophilia; both allo induced which may arise in a haemophilic patient after exposure to clotting factor concentrate, and auto-antibody induced which may arise spontaneously from auto-antibody against coagulation factors.

Further by way of example, an induced coagulation disorder can include hyperfibrinoloysis which may arise after treatment with tissue-type plasminogen activator (tPA).

In one embodiment, the modified histone protein is for the use in the treatment or prevention of haemophilia.

### Treatment or Prevention

As used herein the terms “treat”, “treating” or “treatment” refer to a clinical improvement of a coagulation disorder. Such a clinical improvement may be demonstrated by an improvement of the pathology and/or the symptoms associated with the disease.

In a suitable embodiment an improvement of the symptoms may be demonstrated by reducing the frequency at which the symptoms occur and/or the severity of the symptoms. Merely by way of example, effective treatment may be demonstrated by a reduction in frequency and/or severity of bruising, -reduction in frequency and/or amount of bleeding from injuries, reduction in frequency and/or severity of pain and/or swelling of the joints, reduction in the frequency and/or severity of nose bleeds, and/or severity of menorrhagia.

Clinical improvement of the pathology may be demonstrated, for example, by a reduction in the prothrombin time (PT) and/or the partial thromboplastin time (PTT). Other suitable indications of clinical improvement in the pathology will be known to the skilled person. It will be appreciated that indications of clinical improvement of the pathology will vary depending on the type of coagulation disorder.

The term “prevention” as used herein, refers to prophylactic use of modified histone proteins of the invention. Such prophylactic use may delay or prevent the development of a coagulation disorder and/or bleeding, for example trauma induced bleeding.

Prophylactic use of modified histone proteins may be of particular relevance to an asymptomatic subject at risk of developing a coagulation disorder, for example a subject with a primary disorder which may lead to a secondary coagulation disorder, a subject exposed to environmental factors which lead to a coagulation disorder, and/or a subject known to carry a mutation which increases the subject's likelihood of developing a coagulation disorder.

In one embodiment, the subject may be provided with a modified histone protein as a first line treatment for a coagulation disorder. In such an embodiment, the subject would have not been provided with any other treatment prior to treatment in accordance of the present invention.

However, a modified histone protein may also be used to treat a coagulation disorder in which other treatments were found ineffective. In such embodiments, the subject may have received another treatment prior to treatment in accordance of the present invention. Merely by way of example, the subject may have already received another treatment such as recombinant activated factor VII.

Thus, suitably a modified histone protein may be employed in the use or method of the invention as a second line treatment for a coagulation disorder.

Suitably, the medical use or treatment in accordance with the present invention may make use of the modified histone protein in conjunction with a second treatment. Merely by way of example, a suitable second treatment may be selected from a group consisting of: a chemical; or biopharmaceutical, such as for example, a platelet transfusion, a plasma transfusion, a protein/peptide, an antibody, a nucleic acid etc.

### A Subject

In the context of the methods and medical uses of the present invention, a subject may be one requiring treatment of a coagulation disorder, or preventing a coagulation disorder from developing.

The subject may have symptoms consistent with a coagulation disorder or be asymptomatic.

Symptoms consistent with a coagulation disorder may, for example, include excessive bruising, excessive bleeding from minor injuries, pain and/or swelling of the joints, unusually frequent nose bleeding, and/or menorrhagia.

An asymptomatic subject may be a subject who is believed to be at risk of developing a coagulation disorder. By way of example, a subject at risk of developing a coagulation disorder may have a primary disorder which may lead to a secondary coagulation disorder, be exposed to environmental factors which may lead to an induced coagulation disorder, and/or have a genetic mutation associated with a coagulation disorder.

Suitably, the subject may be a mammal. Merely by way of example, the subject may be a selected from a group consisting of a human, a primate, a dog, a cat, a rat, and a mouse.

In one embodiment, the subject is human. Suitably, the subject may be male or female. Suitably, the subject may be an adult or a child.

### Providing

The term “providing” as used herein encompasses any techniques by which the subject receives a therapeutically effective amount of a modified histone protein of the invention.

In a suitable embodiment, the modified histone protein may be provided to the subject either directly or indirectly.

In an embodiment where the modified histone protein is provided to the subject directly, it may be provided in the form of the protein itself. It will be appreciated that there are various routes in which the subject may be provided with a therapeutically effective amount of the modified histone protein. Such suitable routes may be selected from the group consisting of subcutaneous, intramuscular, intravenous, parenteral, intraperitoneal, intravascular, intranasal, rectal, transdermal, percutaneous and oral. More suitably, the subject may be provided a therapeutically effective amount of the modified histone protein by subcutaneous and/or intramuscular routes. In such an embodiment, the subject may be provided with a pharmaceutical composition comprising the modified histone protein.

In an embodiment where the modified histone protein is provided to the subject indirectly, it may be provided in the form of a nucleic acid encoding such a protein. Methods by which the modified histone protein may be indirectly provided to the subject will be known to those skilled in the art. Merely by way of example, a modified histone protein may be provided to the subject with the use of an expression vector comprising a nucleic acid sequence encoding such a protein.

### Therapeutically Effective Amount

The term “therapeutically effective amount” as used herein, refers to the amount of modified histone protein, that when provided to the subject, is sufficient to promote coagulation. Suitably, the therapeutically effective amount may promote coagulation so as to inhibit bleeding.

Suitably, a therapeutically effective amount is an amount of a modified histone protein, which will result in a clinical improvement of symptoms associated with a coagulation disorder and/or bleeding (for example due to trauma).

It will be appreciated that the therapeutically effective amount will vary depending on various factors, such as the coagulation disorder, the subject's body weight, sex, diet and route by which the modified histone protein is provided.

Such a therapeutically effective amount may be provided to the subject in a single or multiple doses. It will be appreciated that a subject with a coagulation disorder, may require multiple doses of the modified histone proteins of the invention, whereas a subject with acute bleeding, for example due to trauma, may only require a single dose.

It will also be appreciated that the therapeutically effective amount will vary depending on the coagulation disorder. By way of example, a subject suffering from a coagulation disorder (for example a chronic coagulation disorder) may require multiple but lower doses over an extended period of time. On the other hand, a subject suffering from severe bleeding caused by trauma, may require, a single, but high dose of the modified histone proteins. The skilled person will recognise that the therapeutically effective amount will vary depending on the severity of the coagulation disorder.

Such a therapeutically effective amount may be provided to the subject in a single or multiple doses or by continuous infusion. Suitably, the therapeutically effective amount of the modified histone protein is provided to the subject in multiple doses. Suitably, the therapeutically effective amount of the modified histone protein is provided to the subject once every month, twice every month, once every two weeks, once every week, once every few days, once every two days, once per day, more than once per day, twice per day, three times per day, four times per day, once every few hours, once every 6 hours, once every 5 hours, once every 4 hours, once every 3 hours, once every 2 hours, or once every hour.

In one embodiment, the therapeutically effective amount of the modified histone protein is provided to a subject once every four hours.

In a suitable embodiment, the therapeutically effective amount of a modified histone protein may be approximately 0.1, 0.5, 0.6, 0.7, 0.8, 0.9, 1, 5, 10, 15, 20, 30, 40, 50, 60, 70, 80, 90, 100, 120 mg/kg/day or more.

In a suitable embodiment, the therapeutically effective amount of a modified histone protein may be between 0.1 and 120, between 1 and 100, between 10 and 80, or between 20 and 70 mg/kg/day. It will be appreciated that a therapeutically effective amount of a modified histone protein, may be determined in vitro or in vivo, using techniques known to the skilled person.

### An Expression Vector

As touched upon elsewhere in this specification, the modified histone protein may be provided to the subject with the use of an expression vector comprising a nucleic acid sequence encoding such a protein.

Accordingly, the sixth aspect of the invention relates to an expression vector comprising a nucleic acid sequence encoding a modified histone protein.

In a suitable embodiment the expression vector may comprise a nucleic acid encoding amino acid sequence of SEQ ID NO: 6.

Suitably, the expression vector may comprise a nucleic acid encoding an amino acid sharing at least 70% identity with SEQ ID NO:6, at least 75% identity with SEQ ID NO:6, at least 80% identity with SEQ ID NO:6, at least 85% identity with SEQ ID NO: 6, at least 90% identity with SEQ ID NO:6, at least 91% identity with SEQ ID NO: 6, at least 92% identity with SEQ ID NO: 6, at least 93% identity with SEQ ID NO:6, at least 94% identity with SEQ ID NO:6, or at least 95% identity with SEQ ID NO:6. Suitably, the expression vector may comprise a nucleic acid sharing at least 96% identity with SEQ ID NO: 6, at least 97% identity with SEQ ID NO:6, at least 98% identity with SEQ ID NO: 6, or at least 99% identity with SEQ ID NO:6.

In a suitable embodiment the expression vector may comprise a nucleic acid encoding amino acid sequence of SEQ ID NO: 7.

Suitably, the expression vector may comprise a nucleic acid encoding an amino acid sharing at least 70% identity with SEQ ID NO:7, at least 75% identity with SEQ ID NO:7, at least 80% identity with SEQ ID NO:7, at least 85% identity with SEQ ID NO:7, at least 90% identity with SEQ ID NO:7, at least 91% identity with SEQ ID NO:7, at least 92% identity with SEQ ID NO:7, at least 93% identity with SEQ ID NO:7, at least 94% identity with SEQ ID NO:7, or at least 95% identity with SEQ ID NO:7. Suitably, the expression vector may comprise a nucleic acid sharing at least 96% identity with SEQ ID NO:7, at least 97% identity with SEQ ID NO:7, at least 98% identity with SEQ ID NO: 7, or at least 99% identity with SEQ ID NO: 7.

In the context of the sixth aspect, the expression vector may be viral or non-viral. More suitably, the expression vector is viral. By way of example, a suitable viral expression vector may be derived from a virus selected from the group consisting of paramyxovirus, retrovirus, adenovirus, lentivirus, pox virus, alphavirus, and herpes virus. More suitably, the virus is paramyxovirus. An example of a particularly suitable paramyxovirus is Sendai virus. Other viral expression vectors suitable for providing the modified histone protein to the subject are known in the art.

Suitable non-viral expression vectors may be selected from the group consisting of inorganic particle expression vectors (such as calcium phosphate, silica, and gold), lipid based particle expression vectors (for example cationic lipids, lipid nano emulsions, and solid lipid nanoparticles) and polymer based particle expression vectors (for example peptides, polyethylenimine, chitosan, and dendimers). Other suitable non-viral expression vectors will be known to those skilled in the art.

Methods of delivering expression vectors to a cell are also well known in the art. Merely by way of example, such methods include viral transfection, electroporation and sonoporation.

### Coagulation Assay

In accordance with the third aspect, a modified histone protein of the invention may be for use in promoting coagulation in a coagulation assay.

In a suitable embodiment, the modified histone protein for use in promoting coagulation in a coagulation assay does not have reduced cytotoxicity as compared to a corresponding wild-type histone protein.

In an alternative embodiment, the modified histone protein for use in promoting coagulation in a coagulation assay has reduced cytotoxicity as compared to a corresponding wild-type histone protein.

It will be appreciated that in the context of the third aspect, the use of a modified histone protein may have a wide number of applications.

As mentioned elsewhere in this specification, the therapeutically effective amount of the histone protein of the invention may vary depending on factors, such as the coagulation disorder, the subject's body weight, sex, and/or diet.

Accordingly, a coagulation assay which involves the use of a modified histone protein to promote coagulation may be used for the determination of a therapeutically effective amount of the modified histone protein to be provided to the subject. It will be appreciated by the skilled person that such a coagulation assay may be especially informative for the purposes of determining a therapeutically effective amount if it is performed on a blood and/or plasma sample from the subject.

In another example, a coagulation assay which involves the use of a modified histone protein to promote coagulation may be used for monitoring effectiveness of an anti-coagulant therapy in a subject. In particular, such a coagulation assay may be useful for monitoring effectiveness of an anti-coagulant therapy in a subject having a coagulation disorder such as anti-phospholipid antibodies (e.g. lupus anticoagulant). Monitoring the effectiveness of an anti-coagulant therapy in such a subject is currently difficult due to the interaction of the anti-phospholipid antibodies with the phospholipids found in the reagents required for traditional assays such as INR and PTT. As a result of this interaction the availability of phospholipids for clot formation is reduced, which may result falsely in extending clotting time. A coagulation assay which involves the use of a modified histone protein may not require the presence of phospholipids, which may allow clotting time to be measured more accurately.

In one embodiment, there is provided a method of promoting coagulation in a coagulation assay comprising the following steps:
(a) Providing a modified histone protein of the invention in a coagulation assay.

Suitably providing the modified histone protein in the assay may comprise adding the modified histone protein to a sample to be tested for coagulation. Suitably the sample is a blood and/or plasma sample from a subject. Suitably an effective amount of the modified histone protein Is added.

Suitably providing the modified histone protein in the assay may comprise adding the modified histone protein to a sample to be tested for the effectiveness of an anti-coagulant therapy being received by a subject, or to a sample for the determination of a therapeutically effective amount of the modified histone protein to be provided to a subject.

Suitably, therefore, providing the modified histone protein in the assay may comprise adding the modified histone protein to a sample from a subject receiving an anti-coagulant therapy, or adding the modified histone protein to a sample from a subject requiring a pro-coagulant therapy.

Suitably, the method may further comprise a step of (b) measuring the coagulation in the sample. Suitably, measuring coagulation may comprise measuring clotting time of the sample. Suitable means of measuring coagulation and clotting time are exemplified herein.

Suitably the method may further comprise a step of (c) determining the effectiveness of an anti-coagulant therapy in a subject.

Suitably, the method may further comprise a step of (c) determining a therapeutically effective amount of the modified histone protein to be provided to a subject.

Suitably the determining step may be carried out with reference to, and suitably by analysing, the results of step (b).

Suitably these steps of the method may equally be applied to the use defined in the third aspect.

The invention will now be described with reference to the following non-limiting examples.

### EXAMPLES

### Example 1

### 1. Introduction

Thrombin generation is pivotal in the haemostatic response to injury. However, in critical illnesses associated with significant cell injury such as sepsis and trauma, increased thrombin generation is associated with the development of multiple-organ failure (MOF) and the risk of mortality. A key event in thrombin generation is the assembly of the prothrombinase complex involving prothrombin, activated Factors X (FXa) and V (FVa) in the presence of calcium and phospholipids. Prothrombin is a single chain protein composed of fragment 1 (F1), fragment 2 (F2) and a protease domain (prethrombin-2, containing an A and B-chain) that circulates at a concentration of 1.4 μM. F1 and F2 interact with FVa to undergo conformational changes that facilitate cleavage by FXa to generate active thrombin. FVa and anionic phospholipid surfaces accelerate the rate of FXa-mediated prothrombin activation by about 300,000 fold to amplify and localize clot formation to the site of injury.

Generated thrombin can have multifunctional properties but is immediately pro-coagulant through converting fibrinogen into fibrin and amplifying the coagulation cascade by activating FV, FVIII, IX, XI and platelets. Currently recognized mechanisms of increased coagulation activation during critical illness include direct damage to the endothelium and platelets. NETs are extracellular networks of fibres released from neutrophils that contain DNA decorated with antimicrobial proteins and histones. While histones are typically intra-nuclear in forming the basis of chromatin, they become important in host defence when released extracellularly upon cell damage and/or NETosis. Recognized as damage-associated molecular patterns, extracellular histones are directly toxic to invading pathogens but also to host cells when in excess. In vitro, histones have been found to induce thrombin generation in platelet rich plasma (PRP) and inhibit TM-dependent protein C activation, whilst histone infusion in mouse models cause significant cytotoxicity with dose-dependent increases in thrombin-antithrombin complex (TAT) levels, pro-thrombotic consequences and mortality as well as depletion of platelets. In critically ill patients, circulating histone levels also become significantly high and correlate with TAT, development of MOF and increased mortality.

A more direct action of histones has also been proposed. Barranco-Medina et al. showed that H4 and H3 can directly bind prothrombin and cause auto-activation into thrombin under buffer conditions. However, this auto-activation takes up to 8 hours to occur and was not demonstrated in plasma. Its clinical significance remains unclear albeit raising the possibility that histones could directly induce thrombin generation. We demonstrate a novel mechanism whereby histones can substitute for FVa in the prothrombinase reaction without requiring FVa and phospholipid surfaces. This novel system could prime coagulation by generating sufficient thrombin initially to activate FV into FVa and enable classical prothrombinase to proceed.

### 2. Methods

### 2.1 Human Blood Preparation

Peripheral blood was drawn into syringes containing one tenth volume of 0.105M sodium citrate from healthy volunteers after written consent in accordance to protocol approved by Liverpool University Interventional Ethical Committee (Ref: RETH000685). After centrifugation for 20 minutes at 2600 g and 20° C., the resulting platelet poor plasma (PPP) was separated and stored at −80 C. Factor-deficient plasmas were purchased from Affinity Biologicals (Ancaster, Canada).

2.2 Isolation of Histone Binding Proteins from Plasma and Mass Spectrometry Analysis

Citrated PPP was diluted with 2× phosphate buffered saline (PBS) (v/v) and centrifuged to eliminate insoluble material. Harvested supernatant was then pre-cleared using blank Sepharose resin to remove any non-specific protein binding and then loaded onto a CNBr-activated Sepharose 4B column (GE Healthcare, Buckinghamshire, UK) conjugated with calf thymus histones (Roche, West Sussex, UK). After stringent washing with PBS+0.5% (v/v) Tween-20 (Sigma-Aldrich, Dorset, UK) followed by PBS, histone-binding proteins were eluted using 0.1 mol/L glycine (pH 3.5) and then separated using two-dimensional (2D) gel electrophoresis. Protein spots visualized by Coomassie brilliant blue staining were excised and digested with trypsin (Promega, Madison, USA). Resultant peptides were analyzed on a Velos orbitrap mass spectrometer coupled with a Dionex Ultimate 3000 RS. Raw data was processed using PEAKS 7 software against UniProtKB/Swiss-Prot and NCBI databases.

### 2.3 Surface Plasmon Resonance (SPR) Assay

Chips coated with streptavidin (Chip SA, GE Healthcare), which directly interact with histones, were used for immobilizing individual histones and measuring binding affinities with Biocore X-100 system. Binding buffer (100 mmol/L NaCl, 20 mmol/L Tris-HCl, pH 7.4) and regeneration buffer (20 mmol/L HCl) were used throughout the assay. Twenty μg/mL of each recombinant histone (H1, H2A, H2B, H3 or H4) in binding buffer was captured only on the surface of flow cell 2 (Fc2) with Fc1 set as blank. For kinetic analysis, a concentration series of each protein (prothrombin, F1 or F2) was injected at a flow rate of 10 μL/min over both captured histone and reference surfaces (blank) at 20 C. Dissociation constants (Kds) were calculated using software provided by manufacturer.

### 2.4 In Vitro Prothrombin Cleavage and Thrombin Generation

Prothrombin cleavage assay was performed, as previously described, with slight modification. Prothrombin (1.5 μmol/L) (Enzyme Research Laboratories) was first dialyzed against cleavage buffer (20 mmol/L). HEPES, 150 mmol/L NaCl, 5 mmol/L CaCl₂) and incubated with FXa (0.5 nmol/L) (New England Biolabs) in the absence and presence of histones (50 μg/ml). N-Acetyl Heparin (6 μmol/L) (Sigma-Aldrich) or anti-histone single chain variable fragment antibody (ahscFv) (100 μg/mL) were pre-incubated with histones for 10 min in blocking experiments. All experiments were initiated upon addition of prothrombin and terminated by addition of 4× Laemmli buffer.

Active thrombin generation was monitored using chromogenic-based functional assay. Reactions were performed at 37° C. in the presence of calcium (5 mmol/L) and initiated upon addition of S-2238 (250 μmol/L final concentration) (Cambridge Bioscience). Absorbance was continually monitored at 405 nm for 60 min in a Spectromax plate reader. Thrombin activity was calculated as the average rate of S-2238 cleavage (ng/min) within first 10 mins of reaction.

For prothrombinase activity, prothrombin (1.5 μmol/L), FXa (0.5 nmol/L) and procoagulant phospholipids (5 μmol/L), prepared by extrusion, were incubated with either FVa (0.5 nmol/L), H3 or H4 (50 μg/mL) in the presence of calcium (5 mmol/L). Reactions were then terminated at 90 seconds by addition of EDTA (10 mmol/L final concentration) and the total thrombin generated was quantified by S-2238, using commercial thrombin (Enzyme Research Laboratories) as standard.

### 2.5 Computer Modelling

Based on published crystal structures, the interaction between prothrombin (PDB 4HZH) and H2B (from SFUG), H3 or H4 (from 4HGA) were simulated using Docking software (http://zdock.umassmed.edu) to predict the binding models and binding sites. ZDOCK searches all possible binding modes in the translational and rotational space between the two proteins and evaluates each pose using an energy-based scoring function. The model with the highest score in each docking is presented.

### 2.6 Recombinant Prothrombin Fragment 1 and 2 Production

Plasmids for expression of recombinant human prothrombin F1 and F2 with His-tags were synthesized by Invitrogen (Loughborough, UK) and proteins were produced in BL21 bacteria and purified using Ni-NTA resin (QIAGEN, Manchester, UK).

### 2.7 Thrombin Generation and Clot Formation in PPP

Thrombin generation in normal and factor deficient PPP was performed, as previously described. In brief, 80 μL of normal, Factor (F) II, FV or FX-depleted PPP was re-calcified and thrombin generation continuously monitored in a 96-well plate fluorimeter (SpectraMax), using the fluorogenic thrombin substrate z-GGR-AMC (Diagnostica Stago, Reading, UK). Experiments were calibrated against known concentrations of thrombin. For determination of clot formation, reactions were initiated by addition of calcium (12.5 mM final concentration) to PPP and light transmission monitored at 405 nm for 60 min in a 96 well Spectromax plate reader, at 37° C. Clot times were determined by the time taken for 100% light transmission to fully decrease following re-calcification.

### 2.8 Histone-Infusion Mouse Model

Male C57BL/6 mice (8-10 weeks old with body weights of 20-22 g) (Shanghai Laboratory Animal Centre) were kept in the Animal Centre of Southeast University with free access to food and water for 1 week prior to the experiments, that were performed in accordance to National Institute of Health guidelines, under an approved license (Jiangsu province, No. 2151981). Mice (5 per group) were anesthetized with avertin (200 mg/kg) prior to histone infusion with different doses (0, 20, 30, 50, 70 mg/kg) and euthanized 1 h after infusion. Platelet count, prothrombin time (PT), and fibrinogen were performed using clinical biochemistry setup whilst D-dimer and TAT complexes were measured by ELISA (Cusabio). Tissue sections were stained with anti-fibrin antibody (Abcam), as described previously.

### 2.9 Statistical Analysis

Intergroup differences were analysed using ANOVA followed by the Student-Newman-Keuls test. For two-group comparison with and without treatments, Student t-test was used to compare means. Mean±SD are from at least three independent experiments. Spearman rank correlation was used for analysing the correlation of circulating histone levels to DIC scores in septic patients

### 3. Results

3.1 Circulating Histones Directly Interact with Prothrombin

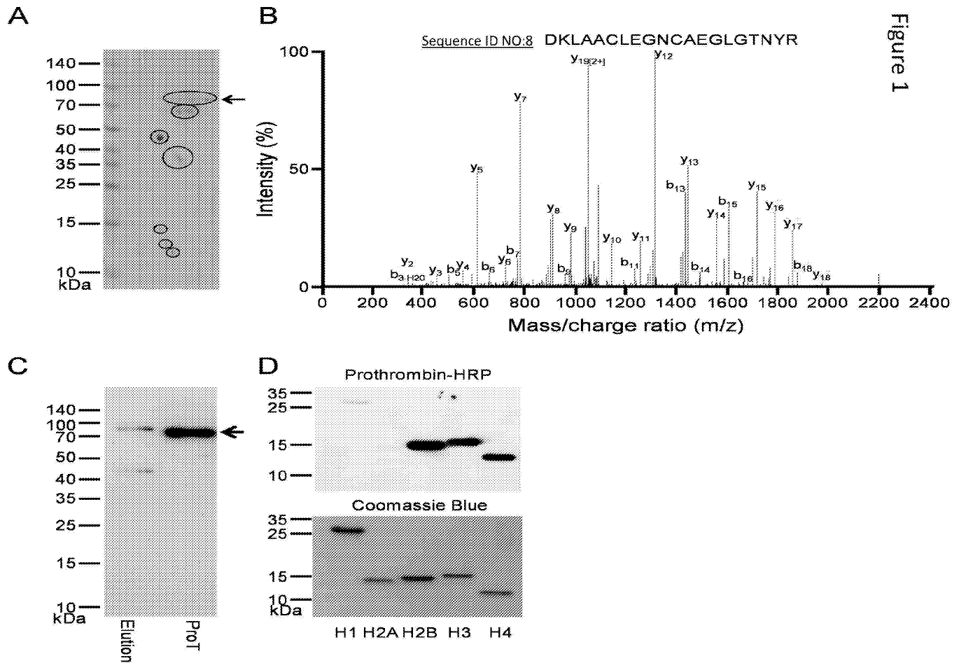

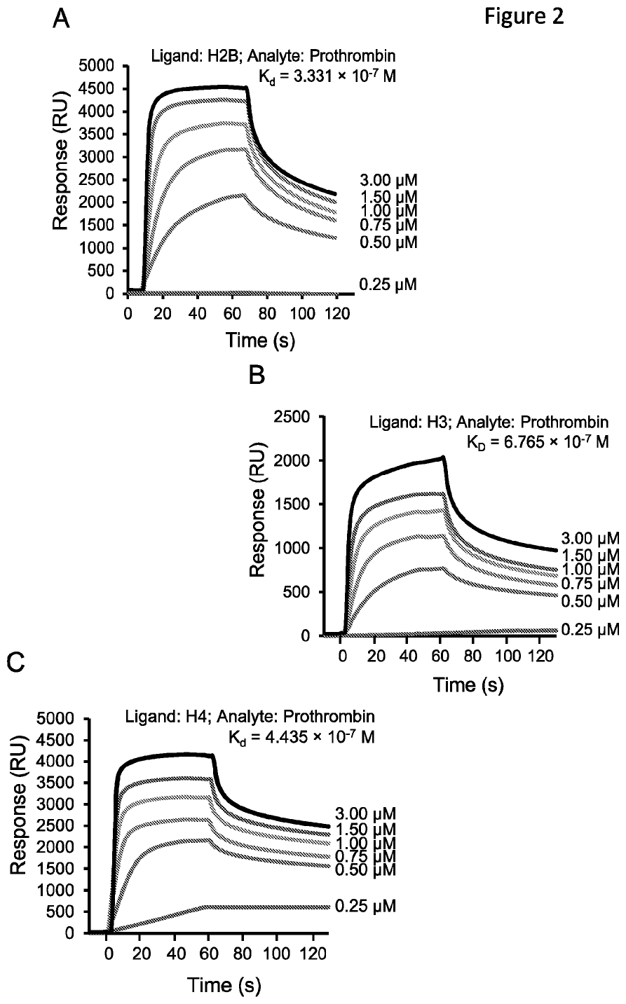

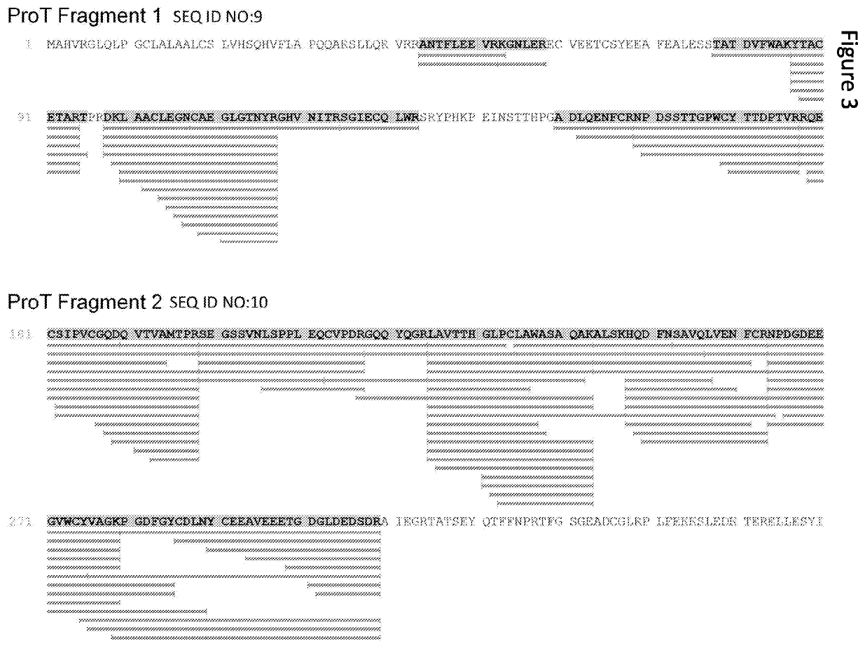

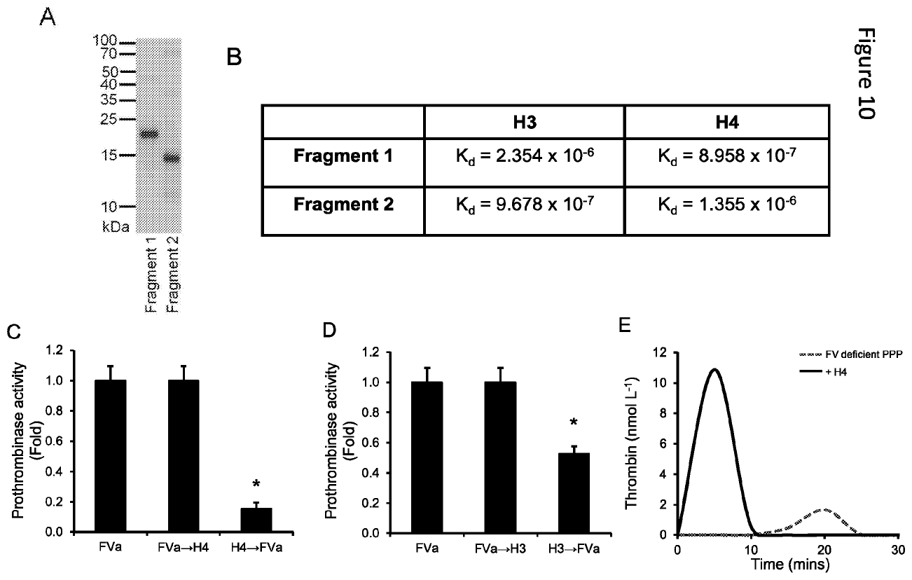

Histone-conjugated Sepharose beads were used to pull down proteins from human plasma. After extensive washing, the proteins pulled down were subjected to 2D gel electrophoresis and seven major histone-binding proteins were visualized by Coomassie blue staining (FIG. 1A). Liquid chromatography-mass spectrometry showed prothrombin as the only coagulation factor identified (FIG. 1B; FIG. 3). This was further confirmed using Western blotting with a specific antibody to prothrombin (FIG. 10). H2B, H3 and H4 showed much stronger interaction with HRP conjugated prothrombin than H2A and H1 (FIG. 1D). Kinetically, H2B, H3 and H4 had Kds of 3.3×10⁻⁷, 6.8×10⁻⁷, and 4.4×10⁻⁷ M respectively in biosensor assay (FIGS. 2A, B and C). The interaction did not require calcium (data not shown) and the Kd values for H1 and H2A were not calculable, due to low binding responses.

### 3.2 Histones Enhance Prothrombin Cleavage and Promote Thrombin Generation by FXa

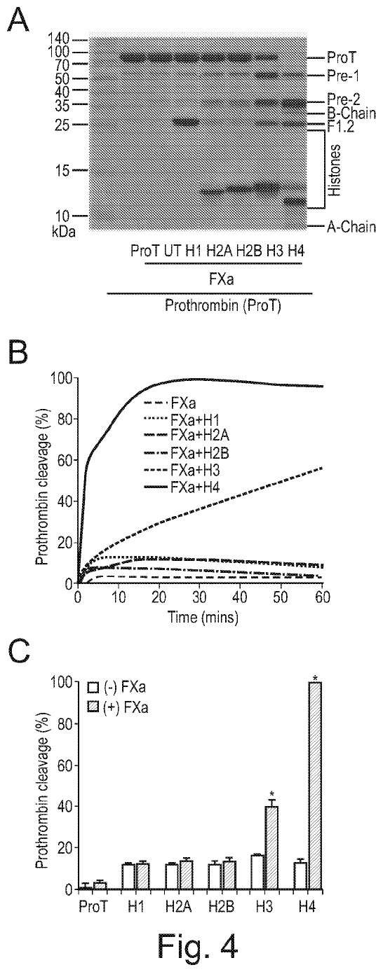

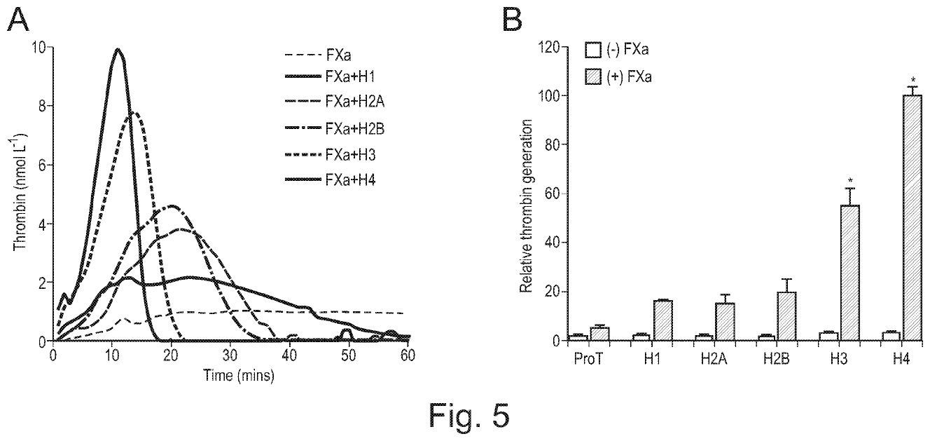

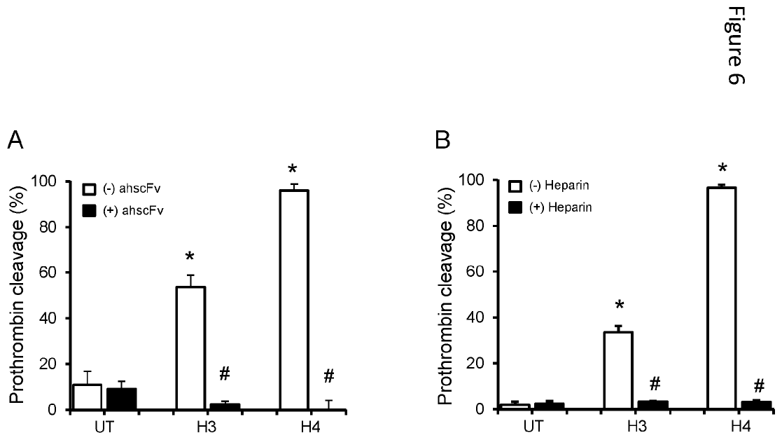

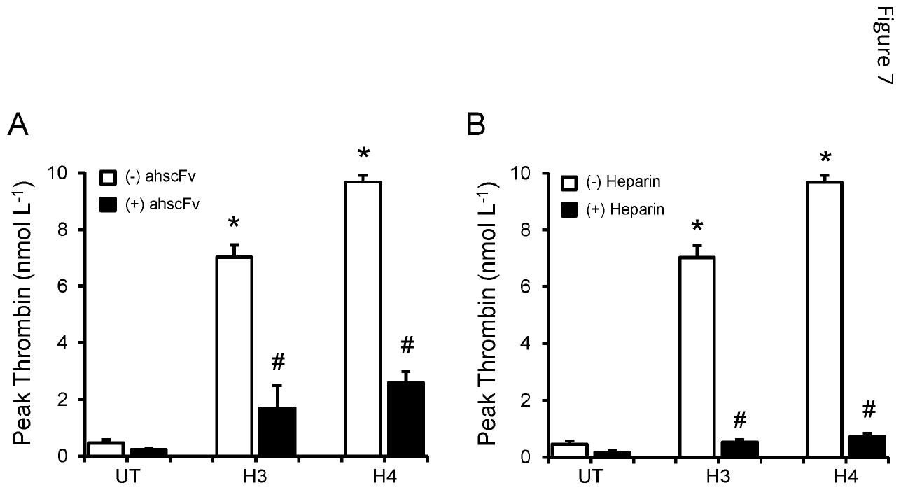

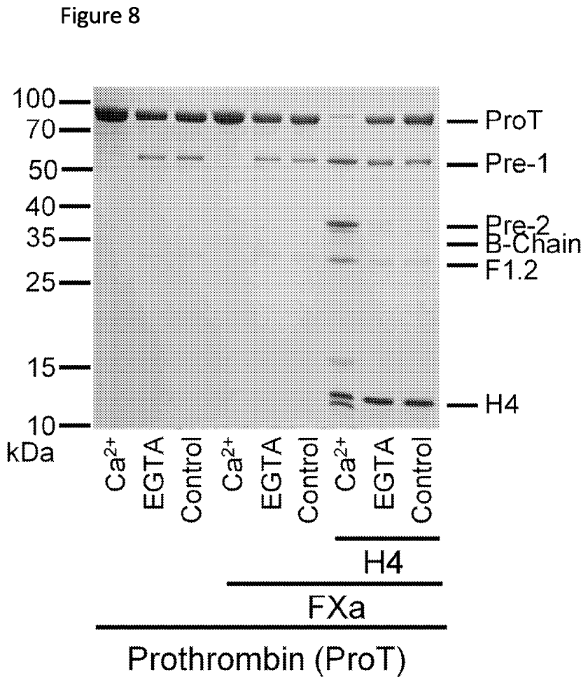

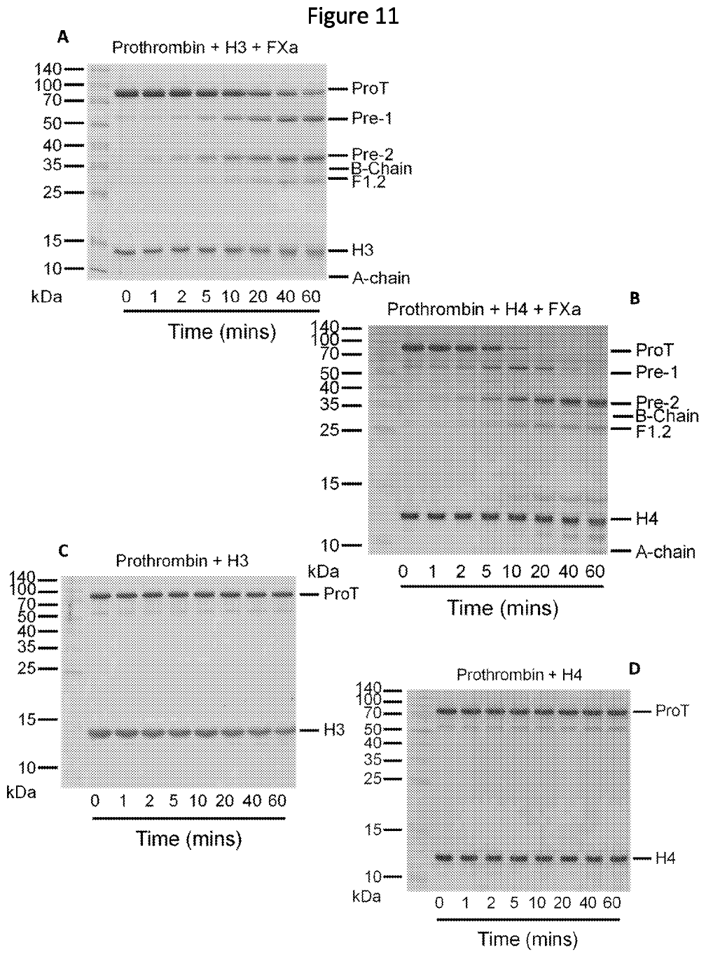

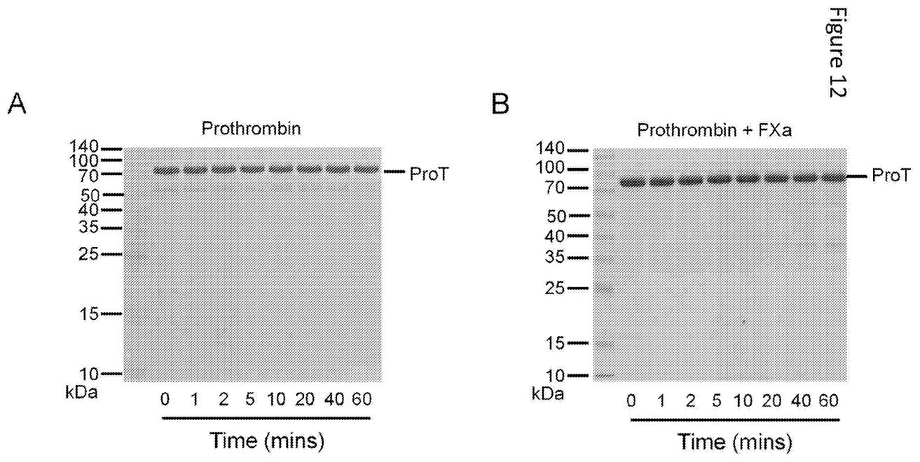

To examine if these interactions had functional consequences, prothrombin cleavage by FXa was performed in the presence or absence of histones. Enhanced calcium-dependent cleavage (FIG. 8) occurred from 1 min to reach significant levels within 1 h in the presence of H3 or H4 but not with the other histones (FIG. 4A-C). The resultant bands include prethrombin 1 (Pre-1) and prethrombin 2 (Pre-2), B-Chain, A-Chain, and F1.2 (FIG. 4A), indicating cleavage of R320 and R271 on prothrombin, which is an essential process in generating thrombin. Indeed, chromogenic thrombin substrate S-2238 was cleaved rapidly by prothrombin and FXa in the presence of H4 or H3 (FIGS. 5A, B). H4 was more effective than H3 in enhancing prothrombin cleavage and thrombin generation with the reaction occurring more rapidly than the reported H4-enhanced prothrombin autoactivation of up to 8 hours. In contrast, H1, H2A and H2B were less effective (FIGS. 4 and 5). AhscFv and heparin as anti-histone reagents could inhibit H3 or H4-enhanced prothrombin cleavage (FIG. 6) and thrombin generation (FIG. 7) to demonstrate that the biological effects was histone-specific. Prothrombin was not obviously cleaved in the absence of FXa (FIG. 4C, FIGS. 11C, D) or histones (FIGS. 12A, B) and no significant thrombin was generated (FIG. 5B). Those data suggest that histones, prothrombin and FXa could form functional complexes with prothrombinase activity.

### 3.3 Histone H3 and H4 Bind Prothrombin Fragment 1/2 to Replace FVa and Assemble an Alternative Prothrombinase Complex

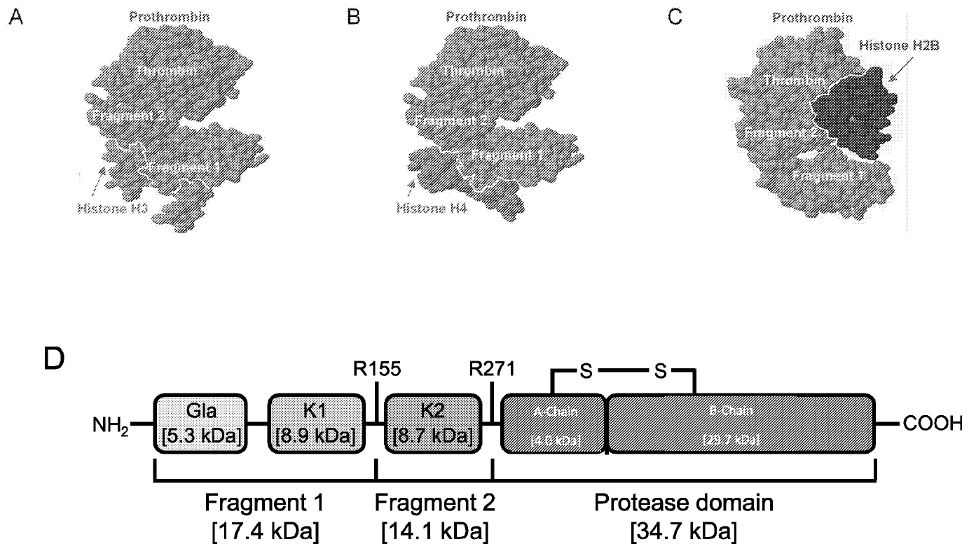

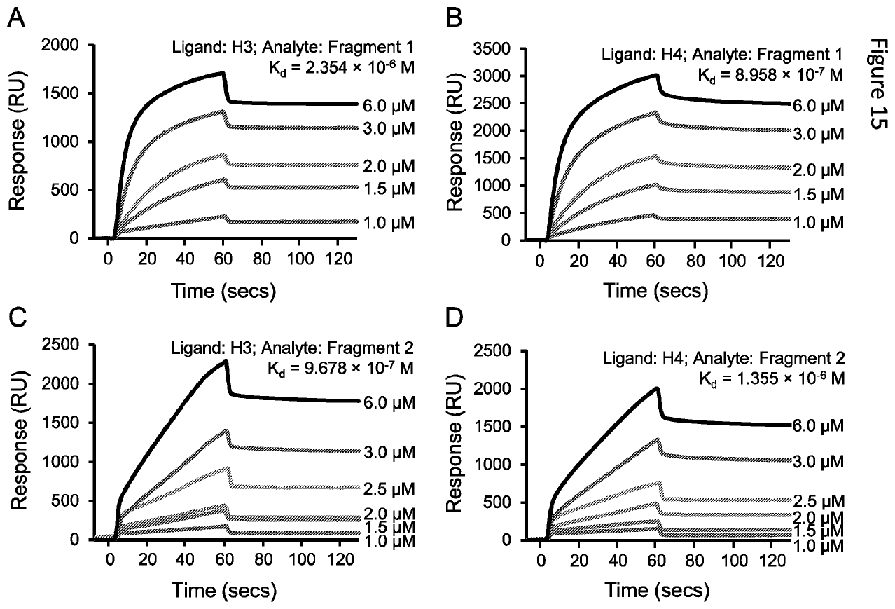

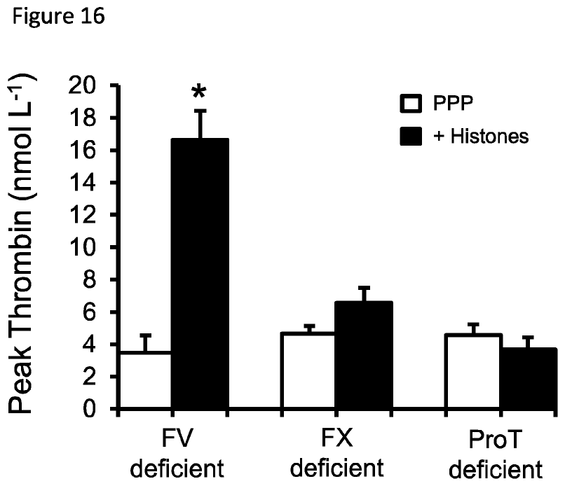

To understand how histones enhance prothrombin cleavage to generate thrombin, their binding sites on prothrombin were investigated. Using docking software, both prothrombin F1 and F2 regions were predicted as the binding sites for H3 or H4 (FIG. 9A, B) whilst the thrombin domain was the predicted binding site for H2B (FIG. 9C). Based on human prothrombin sequence (FIG. 9D), recombinant F1 and F2 peptides was designed and produced in E. coli (FIG. 10A). Using surface plasmon resonance, both F1 and F2 interacted with H3 or H4 with similar affinity (Kds 2.4×10⁻⁶ M-8.9×10⁻⁷ M for F1; Kds 1.4×10⁻⁶ M-9.7×10⁻⁷ M for F2) (FIG. 10B; FIG. 15) as that reported between prothrombin and FVa (Kd ˜2.0×10⁻⁶ M). These data would suggest that both H3 and H4 behave like FVa in co-factor facilitation of prothrombin cleavage by FXa to generate active thrombin. To examine and contrast their relative contributions towards thrombin generation, assessment of prothrombinase activity found no synergism of effects when FVa and H3/H4 were combined (FIG. 100). When H3/H4 was added before FVa, thrombin generation was significantly reduced compared with FVa alone or if histones were added after FVa. This suggests that H3 or H4 could compete with FVa for prothrombin activation into thrombin although the histone-FXa complex is less effective in generating thrombin than the assembly of FVa-FXa. When H4 was added to prothrombin in the presence of FXa and phospholipid mix, consequent addition of FVa could not further increase prothrombinase activity and vice versa (FIG. 100). However, adding FVa after H3, the activity could be significantly increased but did not reach the levels of the classical prothrombinase activity, although adding H3 after FVa could not change the activity (FIG. 10D). H4, but not H3, could fully occupy the FVa binding sites. Addition histones to FV deficient plasma did restored thrombin generating capacity, without supplementation with FVa (FIG. 10E, FIG. 16), indicating H3 or H4 could substitute FVa to form active prothrombinase. To distinguish it from FVa-formed classical prothrombinase, the complex is termed as alternative prothrombinase in this study.

### 3.4 Alternative Prothrombinase, Unlike the Classical One, is Phospholipid-Independent

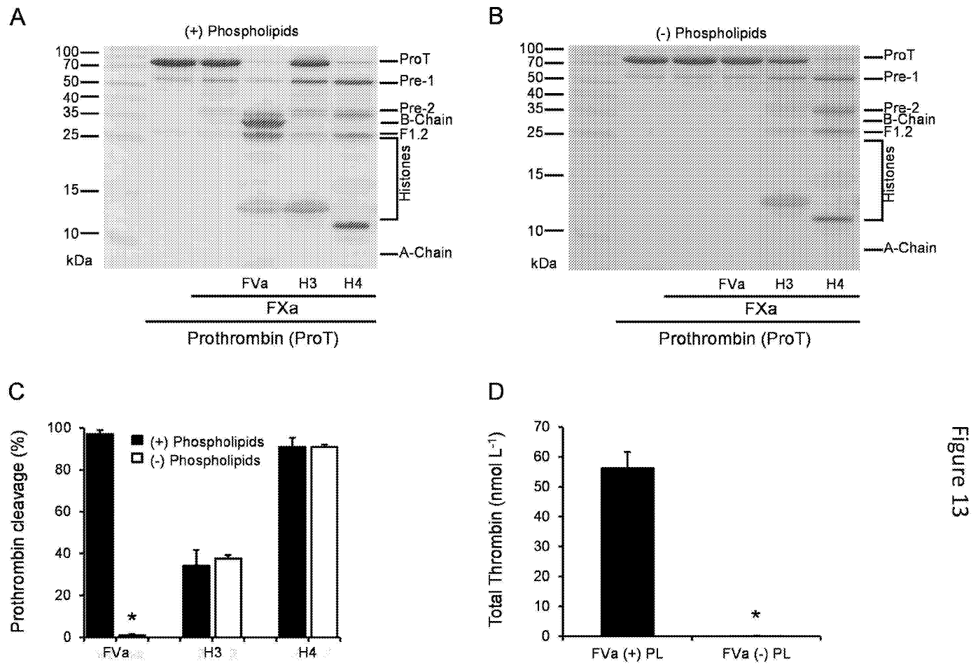

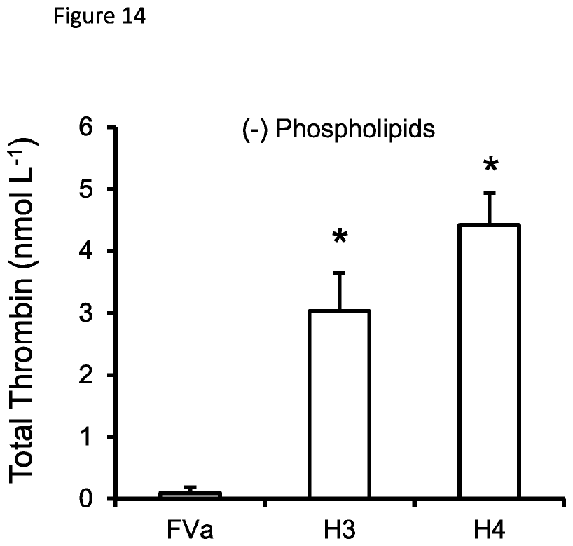

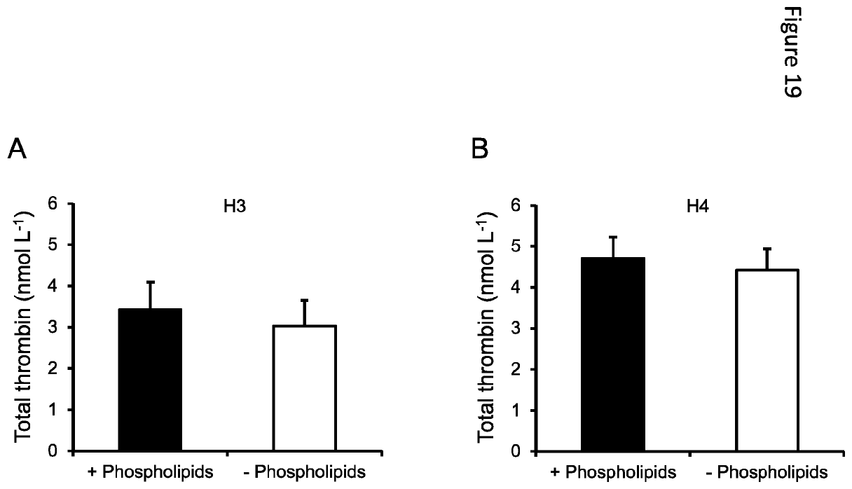

Phospholipids are essential for prothrombin cleavage by FVa-assembled prothrombinase (FIGS. 13A-C) and subsequent thrombin generation (FIG. 13D), which is consistent with published reports. 56.24±5.42 nmol/L thrombin was generated in 90 seconds in the presence of phospholipids (p<0.001). In their absence, prothrombin cleavage was not detectable with only trace amounts of thrombin generated (0.095±0.094 nmol/L in 90 seconds) (FIG. 13D, FIG. 14). However, the presence or absence of phospholipids had no effect on prothrombinase activity or prothrombin cleavage when FVa was substituted by H4 (4.71±0.52 vs 4.42±0.52 nmol/L, p>0.05) or H3 (3.43±0.66 vs 3.03±0.62 nmol/L, p>0.05) (FIG. 19; FIG. 13).

In the absence of phospholipids, H4 and H3 were much more effective in facilitating active thrombin generation than FVa (FIG. 14). These observations suggest that unlike FVa, H4 and H3 enhancement of prothrombinase is not dependent on phospholipid surface availability or localization to the site of injury and could therefore contribute to systemic thrombin generation DIC when histone concentrations are elevated in the circulation.

### 3.5 the Alternative Prothrombinase Initiates Clotting Requiring Much Less FXa and Enables Haemophilic Plasma Clotting

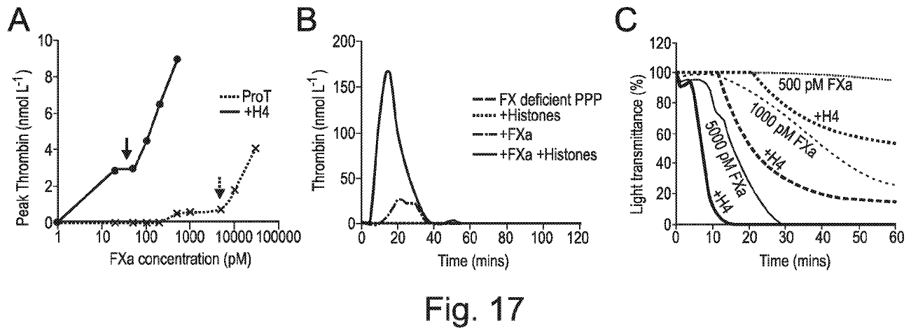

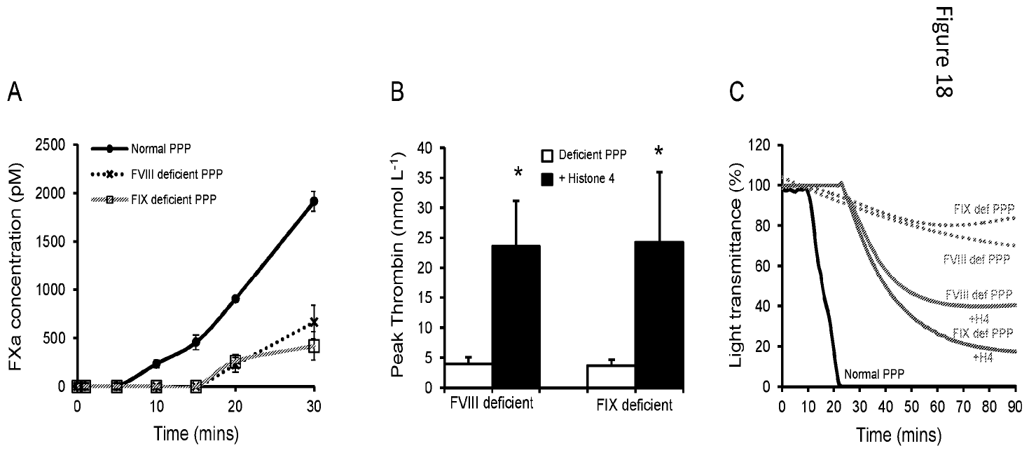

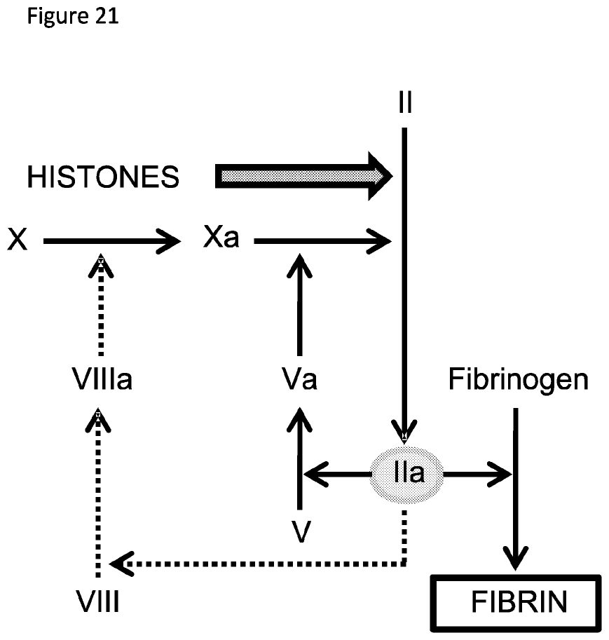

Although high concentration of FXa could directly generate FVa and thrombin to amplify the clotting process, only very low levels (Pico molar) of FXa is produced in the initiation phase. It is still a paradox whether this concentration is sufficient to generate critical levels of thrombin and FVa to initiate clotting and therefore some important factor appears still missing in the process. To explore whether histone exposure plays important roles in the initial phase prior FV activation, different concentrations of FXa were tested in both thrombin generation and clotting assays in the presence or absence of histones. We found that significant thrombin generation only occurred when FXa reached about 1000 pM in the absence of both histones and FVa, whist only 50-100 pM of FXa was required in the presence of 50 μg/ml histone H4, about 1000 times less FXa required (FIG. 17A). Using FX deficient plasma, we could control the concentration of FXa by adding exogenous FXa. No detectable thrombin was generated after re-calcifying the deficient PPP. When 500 pM FXa was supplied, thrombin generation was significant faster in the presence of 50 μg/ml H4 than that without histones (FIG. 17B). To achieve a proper clotting by re-calcifying the PPP, only 500 pM FXa was required in the presence of 50 μg/ml H4, whilst approximately 5000 pM FXa was required in the absence of histones (FIG. 17C). At this concentration, H4 caused much more rapid clotting (within 10 min) than that without histones (over 20 min) (FIG. 17C). Those data suggest that the presence of histones makes clotting initiation much easier and faster. FXa production was amplified through intrinsic pathway after initiation stage in order to generate sufficient FXa required by clot formation. However, this pathway is disabled in haemophilia due to lack of FVIII or FIX and consequently insufficient FXa was produced and led to bleeding (FIG. 21). The levels of FXa in FVIII or FIX deficient plasma (˜200 pM) only reached to less than a quarter of that in normal plasma (˜1000 pM) 20 min after re-calcification (FIG. 18A). As a result, very low concentration of thrombin was generated in the deficient plasmas unless histones were added. Thrombin generation dramatically increased with 50 μg/ml H4 (FIG. 18B). Re-calcifying normal plasma normally led to clot formation, but FVIII or FIX deficient plasma did not clot even increasing incubation time (FIG. 18C). Once adding 50 μg/ml histone H4, re-calcification could result in proper clot formation of the deficient plasmas. Those data confirmed that histones enable much more efficient thrombin generation by low levels of FXa to enable the clotting of haemophilia plasmas.

### 3.6 Prothrombin F1 and F2 Reduce Histone-Induced Systemic Coagulation Activation In Vivo

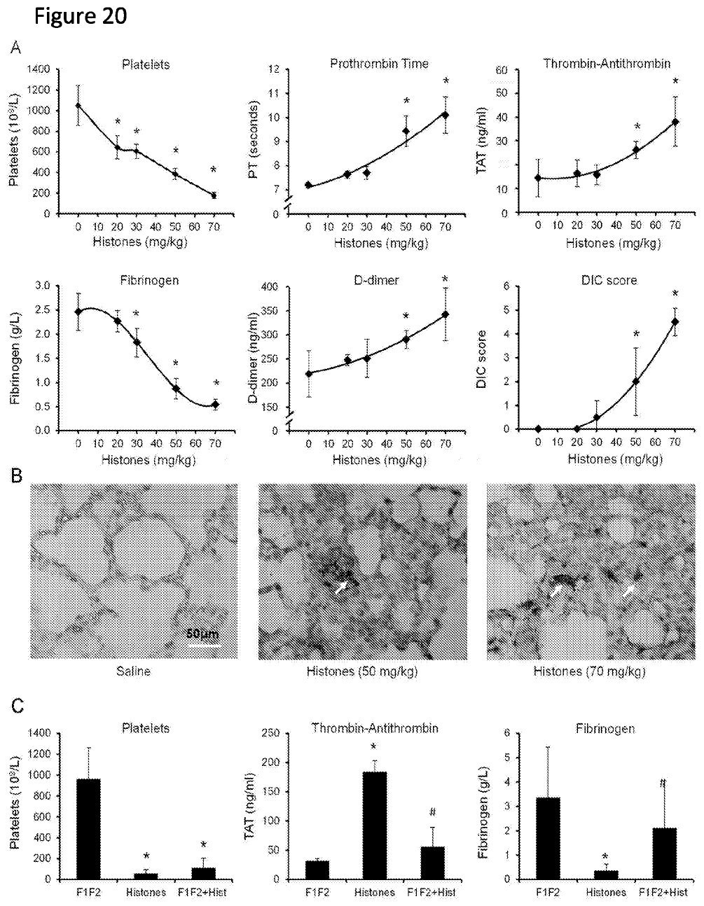

Coagulation disorders are common in critical illness and often lead to DIC. Circulating histones, a pro-coagulant factor, often significantly elevated in those patients. To explore the role of circulating histones in DIC, different concentrations of histones were infused into mice. We observed significant falls in the platelet count and fibrinogen, and elevation in TAT, D-dimer and prothrombin time (PT) in a dose-dependent manner (FIG. 20A), strongly indicating elevated thrombin generation and fibrin clotting formation. Indeed, intravascular thrombi formed and could be easily observed in different organs, particularly in lungs (FIG. 20B). Consistent with the findings of others, histones (over 30 mg/kg) can induce DIC in mice. Using 10 mg/kg prothrombin F1 and F2 5 min prior to 60 mg/kg histone infusion, the platelet count was similar to that of histone-infused mice and no improvement was observed (FIG. 20C), indicating the fragments are unable to neutralizing histone cytotoxicity. In contrast, the histone-induced elevation of TAT was significantly suppressed and histone-induced fibrinogen consumption was significantly reduced (FIG. 20C, middle and right), indicating those fragments, as decoy of prothrombin, strongly suppressed histone-induced thrombin generation. In patients with sepsis (n=129), elevated histone levels in the circulation also correlated with the development of DIC (r=0.446, p<0.0001). Those data support that histone-formed alternative prothrombinase is a very powerful mechanism in promoting systemic coagulation.

### 4. Discussion

Histones are nuclear proteins and can be exposed by damaged blood vessels or released into circulation after cell death or NETosis. Our current finding provides a novel and major mechanism for histone-enhanced coagulation in pathological, and even in physiological and therapeutic contexts. Regulated thrombin generation is pivotal in localizing the haemostatic response to injury but sustained generation and dysregulation can lead to systemic dissemination, which plays an important role in the pathogenesis of critical illness and in worsening disease severity. Typically, classical prothrombinase assembly occurs at altered or injured phospholipid surfaces, e.g. activated platelets or damaged endothelial cell membranes but not on normal cell membranes or in solution. Such a localizing mechanism of the coagulant process serves to prevent dissemination, minimize dysregulation and the chance of deleterious consequences. However, our findings raise the possibility that the phospholipid-independent mechanism could be present. In particular, significant elevation of circulating histones, such as during severe trauma, sepsis or pancreatitis, could form alternative prothrombinase in circulation to generate thrombin and lead to systemic activation of coagulation. Our findings that infusion of histones into mice leads to DIC and that circulating histone levels strongly correlate with DIC scores in critically ill patients indicate that high circulating levels of histones play an important role in inducing systemic coagulation activation. Histone-initialized alternative prothrombinase without anchoring to altered phospholipid surfaces may be a major mechanism of the disseminated coagulation.

Our findings of histones directly interacting with prothrombin to form alternative prothrombinase together with FXa provide a central mechanism of histone-induced thrombosis because all other mechanisms are indirect by providing phospholipid surface or activating FX. More importantly, the significant reverse of histone-induced TAT production and fibrinogen consumption by co-infusion prothrombin fragment 1 and 2 with histones in mice strongly supports that alternative prothrombinase formation is the major mechanism of histone-enhanced thrombin generation and coagulation activation.

In the initiation phase of clotting, producing thrombin and converting FV to FVa is the crucial event for clotting amplification, but how this is achieved is still an unresolved paradox. Certain proteases have been reported to cleave FV, including neutrophil elastase, FVIIa, FXIa, platelet-localised proteases and calpain but none of them demonstrate its efficacy in clotting initiation. Although picomolar concentration of FXa produced by extrinsic tenase in this phase has been predicted by computing simulation to be able to produce thrombin in the presence of saturating phospholipids, the amount of thrombin may not sufficient to generate enough FVa to accelerate the process until high concentration of FXa becomes available which could directly generate both FVa and thrombin in a sufficient amount. In this study, we demonstrated that in the presence of histones, the demanding of FXa become 100-1000 times lower in initiating thrombin generation and clotting. Significant thrombin was generated by the alternative prothrombinase to reach a critical level sufficient for activating FV and intrinsic pathway to amplify the clotting process. Although there may be not many histones released from injured vessels, the locally exposed histones may be quite concentrated and be able to prime the coagulation cascade by forming alternative prothrombinase. Therefore, histone exposure and alternative prothrombinase formation may be the key to solve the FV activation paradox.

Clinically, the amplification of FXa production is blocked in haemophilia due to lack FVIII and FIX, the key factors of intrinsic pathways. Insufficient FXa production leads to bleeding after injury. However, in the present of histone H4, the non-clotting plasma after re-calcifying clotted although taking a bit longer time than normal plasma. Thrombin generation assay showed very low thrombin generation in haemophilia plasma after re-calcifying, but dramatic increase was observed by adding histone H4. Those data confirmed that less FXa was required for clotting in the presence of histones and also demonstrated a potential of treating haemophilia with histones or modified histone peptides without cytotoxicity.

Collectively, our findings indicate that non-toxic, modified histone peptides derived may be potentially used to promote coagulation, and stop bleeding (for example in haemophilia).

### Example 2

### 1. Methods

### 1.1 Methods of Generation Modified Histone Proteins.

Histone peptides were expressed in C41 (DE3) Escherichia coli, using pET-16b expression vector, purified using Histag resin (Qiagen) and assessed by SDS-PAGE.

### 1.2 In Vitro Thrombin Generation in Pure and Plasma Systems.

Prothrombin ws first dialyzed against cleavage buffer (20 mmol/L HEPES, 150 mmol/L NaCl, 5 mmol/L CaCl₂). Thrombin generation was then performed using prothrombin (1.5 μmol/L) incubated with FXa (0.5 nmol/L) (New England Biolabs) in the absence and presence of histones H3C or H4C-terminal peptides (50 μg/ml). Active thrombin generation was monitored using chromogenic-based functional assay. Reactions were performed in a 96-well plate at 37° C., and initiated upon the addition of calcium (5 mmol/L) and S-2238 (250 μmol/L final concentration) (Cambridge Bioscience, Cambridge, UK). Absorbance was continually monitored at 405 nm for 60 min in a Spectromax plate reader. Upon re-calcification of normal plasma, supplemented with H4N or H4C-terminal peptides (50 μg/ml), thrombin generation was continuously monitored in a 96-well plate fluorimeter (SpectraMax), using the fluorogenic thrombin substrate z-GGR-AMC (Diagnostica Stago, Reading, UK). Experiments were calibrated against known concentrations of thrombin (Enzyme Research Laboratories).

### 1.3 Cell Viability Assay.

Cell viability was assessed using EAhy926 (endothelial cells) following 1 hour incubation with or without histones (20 μg/ml). Flow cytometric analysis of propidium iodide-stained cells clearly separated damage cells compared to untreated controls. Viability of untreated cells was set as 100% for comparison.

### 1.4 Histone H3C and H4C Binding to Prothrombin.

2 μg of prothrombin were subjected to SDS-PAGE and probed with HRP-conjugated H3C or H4C, respectively. Bands were visualized with the Pierce ECL reagent (Thermo Scientific).

### 2. Results

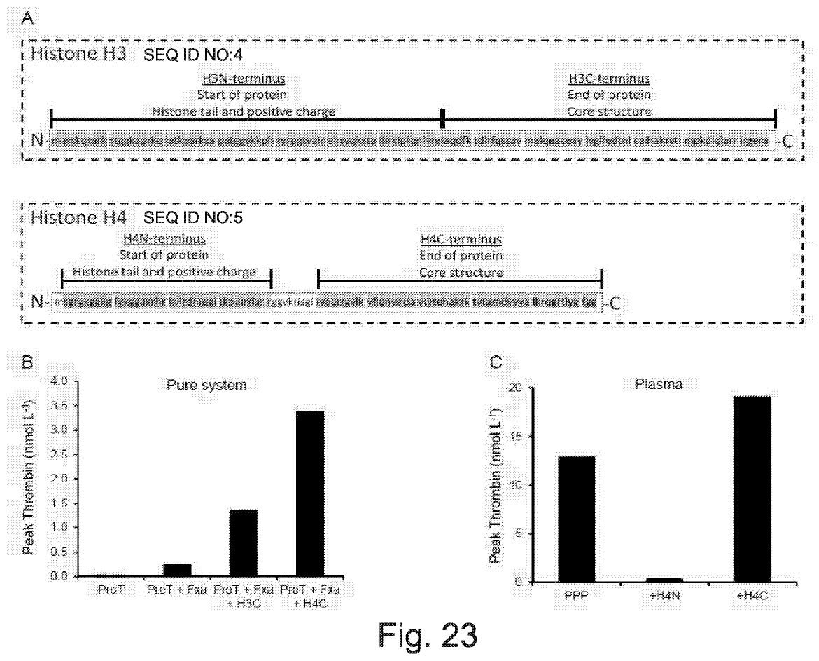

Truncated histones H3C and H4C were found to significantly enhance thrombin generation, in both a pure system (containing prothrombin and FXa), as well as in plasma (see FIG. 23).

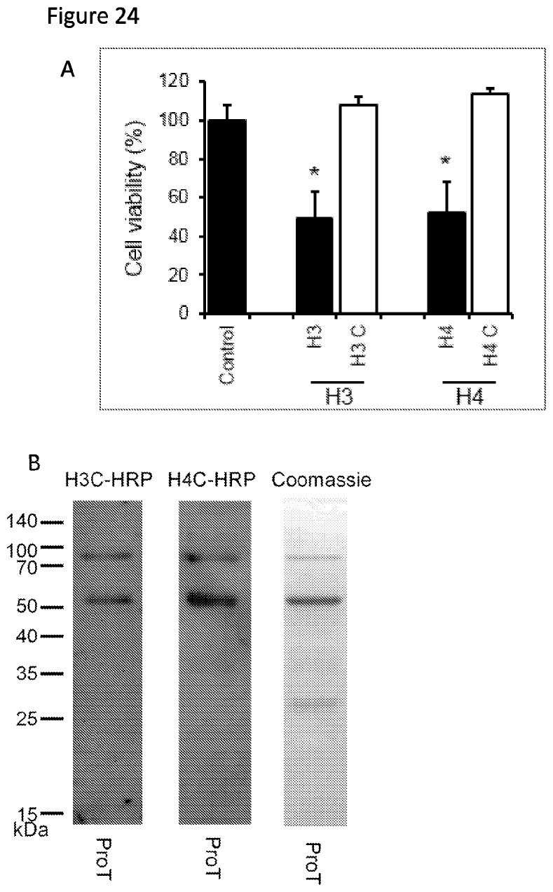

Truncated histones H3C and H4C were found to be non-cytotoxic to cells, unlike their wild-type counterparts (see FIG. 24A).

Using gel overlay assay, truncated histones H3C and H4C were able to bind to prothrombin (see FIG. 24B).

SEQUENCE INFORMATION
Histone H1
SEQ ID NO: 1
MSETAPAETA TPAPVEKSPA KKKATKKAAG AGAAKRKATG
PPVSELITKA VAASKERNGL SLAALKKALA AGGYDVEKNN
SRIKLGLKSL VSKGTLVQTK GTGASGSFKL NKKAASGEAK
PKAKKAGAAK AKKPAGATPK KAKKAAGAKK AVKKTPKKAK
KPAAAGVKKV AKSPKKAKAA AKPKKATKSP AKPKAVKPKA
AKPKAAKPKA AKPKAAKAKK AAAKKK
Histone H2A
SEQ ID NO: 2
MSGRGKQGGK ARAKAKTRSS RAGLQFPVGR VHRLLRKGNY
AERVGAGAPV YLAAVLEYLT AEILELAGNA ARDNKKTRII
PRHLQLAIRN DEELNKLLGK VTIAQGGVLP NIQAVLLPKK
TESHHKAKGK
Histone H2B
SEQ ID NO: 3
MPEPAKSAPA PKKGSKKAVT KAQKKDGKKR KRSRKESYSI
YVYKVLKQVH PDTGISSKAM GIMNSFVNDI FERIAGEASR
LAHYNKRSTI TSREIQTAVR LLLPGELAKH AVSEGTKAVT
KYTSSK
Histone H3
SEQ ID NO: 4
MARTKQTARK STGGKAPRKQ LATKAARKSA PATGGVKKPH
RYRPGTVALR EIRRYQKSTE LLIRKLPFQR LVREIAQDFK
TDLRFQSSAV MALQEACEAY LVGLFEDTNL CAIHAKRVTI
MPKDIQLARR IRGERA
Histone H4
SEQ ID NO: 5
MSGRGKGGKG LGKGGAKRHR KVLRDNIQGI TKPAIRRLAR
RGGVKRISGL IYEETRGVLK VFLENVIRDA VTYTEHAKRK
TVTAMDVVYA LKRQGRTLYG FGG
Modified Histone H3C
SEQ ID NO: 6
IAQDFK TDLRFQSSAV MALQEACEAY LVGLFEDTNL
CAIHAKRVTI MPKDIQLARR IRGERA
Modified Histone H4C
SEQ ID NO: 7
IYEETRGVLK VFLENVIRDA VTYTEHAKRK TVTAMDVVYA
LKRQGRTLYG FGG

1. A modified histone protein for use in promoting coagulation, wherein the modified histone protein has reduced cytotoxicity as compared to a corresponding wild-type histone protein.

2. A modified histone protein for use according to claim 1, wherein the modified histone protein is a fragment of a wild-type histone protein.

3. A modified histone protein for use according to claim 2, wherein the modified histone protein is a fragment of a wild-type histone protein selected from the group consisting of histone H4 and histone H3.

4. A modified histone protein for use according to claim 2 or 3, wherein the fragment consists of approximately 20% to 95%, or 30% to 90%, or 40% to 60%, or 45% to 55% of the total length of the wild-type histone amino acid sequence.

5. A modified histone protein for use according to claims 2 to 4, where the fragment shares at least 70% sequence identity with the corresponding portion of the wild-type histone amino acid sequence.

6. A modified histone protein for use according to claims 2 to 5, wherein the fragment is a truncated wild-type histone protein.

7. A modified histone protein for use according to claims 2 to 6, wherein the fragment lacks at least a part of the N-terminal region.

8. A modified histone protein for use according to claims 2 to 7, wherein the fragment comprises the C-terminal of the corresponding wild-type histone protein.

9. A modified histone protein for use according to claims 2 to 8, wherein the fragment comprises an amino acid sequence sharing at least 70% sequence identity with SEQ ID NO: 6 or SEQ ID NO: 7.

10. A modified histone protein for use according to claim 9, wherein the fragment consists of an amino acid sequence sharing at least 70% sequence identity with SEQ ID NO: 6 or SEQ ID NO: 7.

11. A modified histone protein for use according to any preceding claim, wherein the modified histone protein comprises an amino acid mutation to remove a positively charged amino acid residue.

12. A modified histone protein for use according to claim 11, wherein the positively charged amino acid residue is removed by substitution.

13. A modified histone protein for use according to claim 12, wherein the substitution is with a neutral or negatively charged amino acid residue.

14. A modified histone protein for use according to claims 11 to 13, wherein the positively charged amino acid residue is located in an exposed part of the histone protein structure.

15. A modified histone protein for use according to claims 11 to 14, wherein the positively charged amino acid residue is located in the N-terminal region of the histone protein structure.

16. A modified histone protein for use according to any preceding claim, wherein the modified histone protein has a cytotoxicity of less than 60%, suitably less than 50%, suitably less than 40%, suitably less than 30%, suitably less than 20%, suitably less than 15%, suitably less than 10%, suitably less than 8%, suitably less than 5%, suitably less than 3%, suitably less than 1%, or less as compared to a corresponding wild-type histone

17. A modified histone protein for use according to any preceding claim, wherein the modified histone protein is non-cytotoxic.

18. A modified histone protein for use according to any preceding claim, wherein coagulation is promoted ex vivo or in vivo.

19. A modified histone protein for use according to any preceding claim, wherein coagulation is promoted by upregulating the clotting cascade.

20. A modified histone protein for use according to any preceding claim, wherein the clotting cascade is upregulated by increasing the amount or activity of one or more components of the clotting cascade.

21. A modified histone protein for use in promoting coagulation in inhibition of bleeding, wherein the modified histone protein has reduced cytotoxicity as compared to a corresponding wild-type histone protein.

22. A modified histone protein for use according to claim 21, wherein the modified histone protein is as defined in claims 2 to 17.

23. A modified histone protein for use according to claim 21 or 22, wherein coagulation is promoted as defined by claims 18 to 20.

24. A modified histone protein for use according to claims 21 to 23, wherein bleeding is spontaneous or non-spontaneous.

25. A modified histone protein for use according to claims 21 to 24, wherein bleeding is due to a coagulation disorder.

26. A modified histone protein for use according to claim 25, wherein the coagulation disorder is genetic or acquired.

27. A modified histone protein for use according to claim 25 or 26, wherein the coagulation disorder is caused by a clotting factor deficiency, a clotting protein deficiency, a defective platelet function and/or platelet deficiency, and/or overdevelopment of circulating anticoagulants.

28. A modified histone protein for use according to claim 27, wherein the clotting factor deficiency is selected from the group consisting of haemophilia A, haemophilia B, factor I deficiency, factor II deficiency, factor V deficiency, factor VII deficiency, factor X deficiency, factor XI deficiency, factor XII deficiency and factor XIII deficiency.

29. A modified histone protein for use according to claim 28, wherein the clotting protein deficiency is von Willebrand's disease.

30. A modified histone protein for use according to claim 27, wherein the defective platelet function and/or platelet deficiency is selected from the group consisting of congenital—Bernard Soulier syndrome, Glanzmann's thrombasthenia and platelet storage pool disorder.

31. A modified histone protein for use according to claim 26, wherein the acquired coagulation disorder is a secondary coagulation disorder or an induced coagulation disorder.

32. A modified histone protein for use according to claim 31, wherein the secondary coagulation disorder is cause by a primary disorder selected from the group consisting of liver disease (acquired or genetic), renal disease (acquired or genetic), immune thrombocytopenic purpura, hypergammaglobulinemia (for example multiple myeloma, or Waldenstrom macroglobuliaemia), systemic amyloidosis and vitamin K deficiency.

33. A modified histone protein for use according to claim 31, wherein the induced coagulation disorder is caused by exposure to drugs, alcohol and/or malnutrition.

34. A modified histone protein for use according to claim 33, wherein the drugs are selected from the group consisting of an anticoagulant, an anti-thrombotic, an antibiotic, clopidogrel, and IIb/IIIa inhibitors.

35. A modified histone protein for use according to claim 34, wherein the anticoagulant is selected from the group consisting of warfarin, dabigatran, rivaroxaban, apixaban, and edoxaban.

36. A modified histone protein for use according to claim 34, wherein the anti-thrombotic is aspirin.

37. A modified histone protein for use according to claims 21 to 24, wherein the bleeding is caused by trauma.

38. A modified histone protein for use in promoting coagulation in a coagulation assay.

39. A modified histone protein for use according to claim 38, wherein the modified histone protein is as defined by claims 2 to 17.

40. A modified histone protein for use according to claim 38 or 39, wherein coagulation is promoted as defined by claims 19 to 20.

41. A method of promoting coagulation in a subject in need thereof, the method comprising the step of providing the subject with a therapeutically effective amount of a modified histone protein, wherein the modified histone protein has reduced cytotoxicity as compared to a corresponding wild-type histone protein.

42. A method according to claim 41, wherein the subject is one requiring treatment of a coagulation disorder, or preventing a coagulation disorder from developing.

43. A method according to claim 42, wherein the subject has symptoms consistent with a coagulation disorder or is asymptomatic.

44. A method according to claim 43, wherein symptoms consistent with a coagulation disorder are selected from the group consisting of excessive bruising, excessive bleeding from minor injuries, pain and/or swelling of the joints, unusually frequent nose bleeding, and menorrhagia.

45. A method according to claim 43, wherein an asymptomatic subject is at risk of developing a coagulation disorder.

46. A method according to claims 41 to 45, wherein the modified histone protein is provided directly or indirectly.

47. A method according to claims 41 to 45, wherein the histone is provided directly in the form of a protein.

48. A method according to claims 41 to 45, wherein the histone is provided indirectly in the form of a nucleic acid encoding the modified histone protein.

49. A modified histone protein wherein the modified histone protein is at least 70% identical to one of the following sequences: SEQ ID NO. 6, or SEQ ID NO. 7.

50. A modified histone protein according to claim 49, wherein the modified histone protein consists of one of the following sequences: SEQ ID NO. 6, or SEQ ID NO. 7.

51. A modified histone protein wherein the modified histone protein is at least 70% identical to one of the following sequences: SEQ ID NO. 5, SEQ ID NO. 4, SEQ ID NO. 3, SEQ ID NO 2, and SEQ ID NO. 1.

52. An expression vector comprising a nucleic acid encoding a modified histone protein, wherein the modified histone protein is as defined in any of claims 49-51.


SEQUENCE LISTING (10 sequences)

SEQ ID NO: 1 - protein, 226 aa, Homo sapiens
MSETAPAETATPAPVEKSPAKKKATKKAAGAGAAKRKATGPPVSELITKAVAASKERNGLSLAALKKALAAGGYDVEKNNSRIKLGLKSLVSKGTLVQTKGTGASGSFKLNKKAASGEAKPKAKKAGAAKAKKPAGATPKKAKKAAGAKKAVKKTPKKAKKPAAAGVKKVAKSPKKAKAAAKPKKATKSPAKPKAVKPKAAKPKAAKPKAAKPKAAKAKKAAAKKK

SEQ ID NO: 2 - protein, 130 aa, Homo sapiens
MSGRGKQGGKARAKAKTRSSRAGLQFPVGRVHRLLRKGNYAERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 3 - protein, 126 aa, Homo sapiens
MPEPAKSAPAPKKGSKKAVTKAQKKDGKKRKRSRKESYSIYVYKVLKQVHPDTGISSKAMGIMNSFVNDIFERIAGEASRLAHYNKRSTITSREIQTAVRLLLPGELAKHAVSEGTKAVTKYTSSK

SEQ ID NO: 4 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 5 - protein, 103 aa, Homo sapiens [sequence omitted]

SEQ ID NO: 6 - protein, 62 aa, Homo sapiens
IAQDFKTDLRFQSSAVMALQEACEAYLVGLFEDTNLCAIHAKRVTIMPKDIQLARRIRGERA

SEQ ID NO: 7 - protein, 53 aa, Homo sapiens
IYEETRGVLKVFLENVIRDAVTYTEHAKRKTVTAMDVVYALKRQGRTLYGFGG

SEQ ID NO: 8 - protein, 20 aa, Artificial Sequence
  Histone binding protein (figure 1)
DKLAACLEGNCAEGLGTNYR

SEQ ID NO: 9 - protein, 180 aa, Artificial Sequence
  ProT fragment 1
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQE

SEQ ID NO: 10 - protein, 180 aa, Artificial Sequence
  ProT fragment 2
CSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYI